(19) United States
(12) Patent Application Publication
(10) Pub. No.: US 2024/0164698 A1
(43) Pub. Date: May 23, 2024

(54) HEAD-MOUNTABLE OCULOMOTOR ASSESSMENT DEVICE AND SYSTEM, AND METHOD OF USING SAME

(71) Applicant: EVOLUTION OPTIKS LIMITED, Christ Church (BB)

(72) Inventors: Raul MIHALI, Westport (CA); Khaled EL-MONAJJED, Laval (CA); John Thomas JACOBSEN, Westport, CT (US)

(21) Appl. No.: 18/548,851

(22) PCT Filed: Jan. 24, 2022
(86) PCT No.: PCT/US2022/013564

§ 371 Date: Sep. 1, 2023

Related U.S. Application Data
(60) Provisional application No. 63/179,057, filed on Apr. 23, 2021.
(60) Provisional application No. 63/200,433, filed on Mar. 5, 2021.
(60) Provisional application No. 63/274,873, filed on Nov. 2, 2021.

(51) Int. Cl. A61B 5/00; A61B 3/113; A61B 5/16

(52) U.S. Cl. CPC A61B 5/4064; A61B 3/113; A61B 5/163; A61B 5/6803; A61B 5/7435

(57) ABSTRACT

Described are various embodiments of an oculomotor assessment system and method of using same. One embodiment relates to system comprising a head-mountable device comprising a set of light sources mountable on the user's head to present. in operation, a visual stimulus at a corresponding plurality of physical locations at respective relative distances to the user's eyes. The system further comprises an eye tracking system configured to monitor an oculomotor response of the user to the visual stimulus, and a digital data processor operable to execute digital instructions for performing the oculomotor assessment by activating the set of light sources in a sequence to present the visual stimulus in the corresponding physical locations at the respective relative distances, recording the oculomotor response, and outputting an assessment result indicator as a result of the oculomotor response.

### CROSS REFERENCE TO RELATED APPLICATIONS

This application claims priority to U.S. Provisional Application No. 63/200,433 filed Mar. 5, 2021, U.S. Provisional Application No. 63/179,057 filed Apr. 23, 2021, and U.S. Provisional Application No. 63/274,873 filed Nov. 2, 2021, the entire disclosures of each of which are hereby incorporated herein by reference.

### FIELD OF THE DISCLOSURE

The present disclosure relates to oculomotor assessment, and, in particular, to a head-mountable oculomotor assessment device and system, and method of using same.

### BACKGROUND

The oculomotor system is a relatively accessible portion of the nervous system, wherein abnormalities in its behaviour may serve as biomarkers for a range of conditions. For example, a traumatic brain injury such as a concussion may result in visual disorders related to convergence insufficiency (CI), accommodative insufficiency (AI), and mild saccadic dysfunction (SD). It may also be associated with abnormalities of saccades, pursuit eye movements, convergence, accommodation, and the vestibular-ocular reflex. Accordingly, evaluation of one or more of these aspects may be useful in the assessment of cognitive function of an individual.

Various eye tracking assessment algorithms have been proposed to monitor or diagnose brain injuries. For instance, U.S. patent application Ser. No. 18/0,235,532 entitled ‘Method and System for Detection Concussion’ and published to Samandani, et al. on Aug. 23, 2018 discloses a method for identifying a concussion through the analysis of a subject's blinking as compared to a baseline. Similarly, Oculogica's EyeBOX®, a device marketed for concussion assessment, provides a BOX ScoreSM based on similar eye tracking analysis.

U.S. patent application Ser. No. 19/0,239.790 entitled ‘Systems and Methods for Assessing User Physiology Based on Eye Tracking Data’ and published to Gross and Hunfalvay on Aug. 8, 2019 discloses another example of a method of assessing user physiology based on eye tracking. Such processes may be used to provide reports reflective of potential neurological problems, such as those generated by the RightEye EyeQ™ technology.

Similarly, U.S. Pat. No. 9,004,687 entitled ‘Eye Tracking Headset and System for Neuropsychological Testing Including the Detection of Brain Damage’ and issued to Stack on Apr. 14, 2015 discloses a headset operable to display 2D images for performing smooth pursuit eye tracking exams to indicate potential cognitive impairment. Conversely, U.S. patent application Ser. No. 19/0,082.954 entitled ‘Objective Testing of Vergence Dysfunction for Diagnosis and Vergence Recovery Convalescence Using Dynamic Vergence Testing Platform Including 3D Head Mounted Display System with Integrated Eye Tracking Technology’ published Mar. 21, 2019 to Kiderman and Ashmore discloses a wired head-mounted display to perform vergence dysfunction tests that attempts to simulate a change in perceived object depth using on-screen depth cues.

Further to the notion of employing a headset for the display of visual stimuli in the assessment of a potential cognitive impairment, U.S. Pat. No. 10,719,992 entitled ‘Augmented Reality Display System for Evaluation and Modification of Neurological Conditions, Including Visual Processing and Perception Conditions’ and issued to Samec, et al. on Jul. 21, 2020 discloses an augmented reality display that is further configured as a transcranial doppler device to search for abnormalities that may be indicative of a concussion.

However, various challenges are known to exist with respect to the provision of visual content using augmented reality (AR) and virtual reality (VR) systems. For example, conflicting sensory stimuli experienced by a user of an AR or VR system may lead to user fatigue or nausea. U.S. Pat. No. 10,871,627 entitled ‘Head-Mounted Display Device with Direct-Current (DC) Motors for Moving Displays’ and issued to Fang, et al. on Dec. 22, 2020 attempts to address this issue by coupling a DC motor to a display of an AR or VR system, thereby allowing the display to move during use and mitigate vergence-accommodation conflicts.

This background information is provided to reveal information believed by the applicant to be of possible relevance. No admission is necessarily intended, nor should be construed, that any of the preceding information constitutes prior art or forms part of the general common knowledge in the relevant art.

### SUMMARY

The following presents a simplified summary of the general inventive concept(s) described herein to provide a basic understanding of some aspects of the disclosure. This summary is not an extensive overview of the disclosure. It is not intended to restrict key or critical elements of embodiments of the disclosure or to delineate their scope beyond that which is explicitly or implicitly described by the following description and claims.

A need exists for an oculomotor assessment system, and method of using same that overcome some of the drawbacks of known techniques, or at least, provides a useful alternative thereto. Some aspects of this disclosure provide examples of such systems and methods.

In accordance with one aspect, there is provided a system for performing an oculomotor assessment of a user, the system comprising: a head-mountable device comprising a set of light sources mountable on the user's head to present, in operation, a visual stimulus at a corresponding plurality of physical locations at respective relative distances to the user's eyes, and an eye tracking system configured to monitor an oculomotor response of the user to the visual stimulus. The system comprises a digital data processor in communication with the set of light sources and the eye tracking system and operable to execute digital instructions for performing the oculomotor assessment. The assessment is performed by activating the set of light sources in a sequence to present the visual stimulus in the corresponding physical locations at the respective relative distances in accordance with the oculomotor assessment, recording the oculomotor response, and outputting an assessment result indicator as a result of the oculomotor response.

In one embodiment, the set of light sources comprises a plurality of distinctly operable light sources.

In one embodiment, the plurality of distinctly operable light sources comprises a plurality of light emitting diodes (LEDs).

In one embodiment, the head-mountable device further comprises a digital display screen disposed relative to the set of light sources so to be unobstructively viewable by the user at a set distance from the user's eyes, and wherein the digital display screen is operable to render a complementary two-dimensional oculomotor stimulus at the set distance.

In one embodiment, the set of light sources comprises a set of pixels of a digital display screen, the head-mountable device further comprising one or more actuators in communication with the digital data processor and coupled to the digital display screen, wherein the one or more actuators are operable to displace the display screen so to dispose the set of pixels at the respective relative distances in accordance with the oculomotor assessment.

In one embodiment, the digital display screen is further operable to render a complementary two-dimensional oculomotor stimulus at a set distance.

In one embodiment, the set of light sources comprises corresponding pixel subsets of a display screen, each of the subsets being independently addressable by the digital data processor and corresponding to distinct regions of the display screen, the head-mountable device further comprising a plurality of optical guides, each of the plurality of optical guides corresponding to a respective one of the subsets and disposed relative thereto so to guide light from the respective one of the subsets to a corresponding one of the physical locations to produce the visual stimulus.

In one embodiment, the digital display screen is further operable to render a complementary two-dimensional oculomotor stimulus at a set distance.

In one embodiment, the digital data processor is an onboard processor of the head-mountable device.

In one embodiment, the oculomotor assessment comprises a cognitive impairment assessment.

In one embodiment, the cognitive impairment assessment comprises a vergence response assessment.

In one embodiment, the head-mountable device comprises a widescreen display disposed relative to the set of light sources so to be unobstructively viewable by the user at a set distance from the user's eyes in direct unrefracted line of sight to render a dynamic visual stimulus horizontally displaceable in a wide binocular field of view to stimulate a complementary wide field of view oculomotor response thereto in accordance with a complementary assessment.

In one embodiment, the widescreen display is physically mounted within a viewing tunnel that optically isolates, when mounted up against the user's face, viewing of the widescreen display, and wherein the set of light sources are operatively mounted along at least one of an upper or a lower internal surface of the viewing tunnel along an axis linking the user and the widescreen display.

In one embodiment, the viewing tunnel comprises a substantially amorphous internal surface.

In one embodiment, the eye tracking system comprises at least one tracking light source oriented to illuminated the user's eyes, and at least one camera oriented to capture a response of the user's eyes to illumination from the at least one tracking light source.

In one embodiment, the at least one light source comprises an infrared (IR) light source, and wherein the at least one camera is at least sensitive to IR light.

In one embodiment, the sequence comprises a consecutive linear sequence.

In accordance with another aspect, there is provided a head-mountable device for performing an oculomotor assessment of a user, the device comprising a set of light sources disposable, when the device is mounted on the user's head, to present, in operation, a visual stimulus at a corresponding plurality of physical locations at respective relative distances to the user's eyes, and an eye tracking system configured to monitor an oculomotor response of the user to the visual stimulus.

In one embodiment, the set of light sources comprises a plurality of distinctly operable light sources.

In one embodiment, the plurality of distinctly operable light sources comprises a plurality of light emitting diodes (LEDs).

In one embodiment, the device further comprises a digital display screen disposed relative to the set of light sources so to be unobstructively viewable by the user at a set distance from the user's eyes, and wherein the digital display screen is operable to render a complementary two-dimensional oculomotor stimulus at the set distance.

In one embodiment, the set of light sources comprises a set of pixels of a digital display screen, the head-mountable device further comprising one or more actuators in communication with the digital data processor and coupled to the digital display screen, wherein the one or more actuators are operable to displace the display screen so to dispose the set of pixels at the respective relative distances in accordance with the oculomotor assessment.

In one embodiment, the digital display screen is further operable to render a complementary two-dimensional oculomotor stimulus at a set distance.

In one embodiment, the set of light sources comprises corresponding pixel subsets of a display screen, each of the subsets being independently addressable and corresponding to distinct regions of the display screen, the device further comprising a plurality of optical guides, each of the plurality of optical guides corresponding to a respective one of the subsets and disposed relative thereto so to guide light from the respective one of the subsets to a corresponding one of the physical locations to produce the visual stimulus.

In one embodiment, the digital display screen is further operable to render a complementary two-dimensional oculomotor stimulus at a set distance.

In one embodiment, the oculomotor assessment comprises a vergence response assessment.

In one embodiment, the device comprises a widescreen display disposed relative to the set of light sources so to be unobstructively viewable by the user at a set distance from the user's eyes in direct unrefracted line of sight to render a dynamic visual stimulus horizontally displaceable in a wide binocular field of view to stimulate a complementary wide field of view oculomotor response thereto in accordance with a complementary assessment.

In one embodiment, the widescreen display is physically mounted within a viewing tunnel that optically isolates, when mounted up against the user's face, viewing of the widescreen display, and wherein the set of light sources are operatively mounted along at least one of an upper or a lower internal surface of the viewing tunnel along an axis linking the user and the widescreen display.

In one embodiment, the viewing tunnel comprises a substantially amorphous internal surface.

In one embodiment, the eye tracking system comprises at least one tracking light source oriented to illuminated the user's eyes, and at least one camera oriented to capture a response of the user's eyes to illumination from the at least one tracking light source.

In one embodiment, the at least one light source comprises an infrared (IR) light source, and wherein the at least one camera is at least sensitive to IR light.

In one embodiment, the device further comprises a digital data processor in communication with set of light sources and the eye tracking system and operable to execute digital instructions for performing the oculomotor assessment by: activating the set of light sources in sequence to present the visual stimulus in the corresponding physical locations at the respective relative distances in accordance with the oculomotor assessment; recording the oculomotor response; and outputting an assessment result indicator as a result of the oculomotor response.

In accordance with another aspect, there is provided a head-mountable device for performing an oculomotor assessment of a user, comprising a widescreen display to be disposed, when the device is mounted, in direct unrefracted line of sight to render a dynamic visual stimulus horizontally displaceable in a wide binocular field of view to stimulate a wide field of view oculomotor response thereto, and an eye tracking system configured to monitor the wide field of view oculomotor response.

In one embodiment, the device comprises a digital data processor in communication with the widescreen display and the eye tracking system and operable to execute digital instructions for performing the ocular cognitive impairment assessment by activating the wide screen display to horizontally displace the dynamic visual stimulus in accordance with the oculomotor assessment, recording the oculomotor response, and outputting an assessment result indicator as a result of the oculomotor response.

In one embodiment, the widescreen display is physically mounted within a viewing tunnel that optically isolates, when mounted up against the user's face, viewing of the widescreen display.

In one embodiment, the viewing tunnel comprises a substantially amorphous internal surface.

In one embodiment, the amorphous internal surface is at least partially provided by a fabric.

In one embodiment, the wide binocular field of view comprises a horizontal field of view of at least 65 degrees.

In one embodiment, the horizontal field of view is of at least 70 degrees.

In one embodiment, the device comprises a plurality of light sources disposed to project inwardly from the widescreen display and operable to present a visual stimulus at a corresponding plurality of physical locations at respective relative distances to the user.

In one embodiment, the plurality of light sources is disposed along an upper viewing tunnel surface extending from above the widescreen display toward the user.

In one embodiment, the plurality of light sources is activated to test a near point of convergence.

In one embodiment, the eye tracking system comprises at least one tracking light source oriented to illuminated the user's eyes, and at least one camera oriented to capture a response of the user's eyes to illumination from the at least one tracking light source.

In one embodiment, the at least one light source comprises an infrared (IR) light source, and wherein the at least one camera is at least sensitive to IR light.

In accordance with another aspect, there is provided a

A system for performing an oculomotor assessment of a user, the system comprising: a head-mountable device comprising a widescreen display to be disposed, when the device is mounted, in direct unrefracted line of sight to render a dynamic visual stimulus horizontally displaceable in a wide binocular field of view to stimulate a wide field of view oculomotor response thereto, and an eye tracking system configured to monitor the wide field of view oculomotor response; and a digital data processor in communication with the widescreen display and the eye tracking system and operable to execute digital instructions for performing the ocular cognitive impairment assessment. The assessment is performed by activating the wide screen display to horizontally displace the dynamic visual stimulus in accordance with the ocular cognitive impairment assessment, recording the oculomotor response, and outputting an assessment result indicator as a result of the oculomotor response.

In one embodiment, the system further comprises and an operator application digitally executable on a distinct operator device having a digital display screen and a communication interface to the head-mountable device, wherein the operator application comprises digitally executable instructions to render a graphical user interface (GIU) on the digital display screen and receive as input therefrom manual digital control of the dynamic visual stimulus such that a stimulus displacement on the widescreen display corresponds with a manual displacement entered via the GUI.

In accordance with another aspect, there is provided a head-mountable device for performing an oculomotor assessment of a user, the head-mountable device comprising a head-mountable housing defining an immersive internal visual stimulation chamber therein, the housing further comprising an external assessment indicator, a dynamic visual stimulation system operatively disposed to operate within the immersive internal visual stimulation chamber to render a dynamic visual stimulus to stimulate an oculomotor response thereto, an eye tracking system configured to monitor the oculomotor response, and a digital data processor. The digital data processor is operable to execute digital instructions for performing the oculomotor assessment via the dynamic visual stimulation system and screen for an oculomotor-related health risk based at least on the oculomotor response thereto as monitored via the eye tracking system, and output, via the external assessment indicator, a screening indicator representative of the health risk.

In one embodiment, the external assessment indicator is physically disposed so to be externally perceivable by an individual facing the user upon which the device is mounted.

In one embodiment, the external assessment indicator comprises a colour-coded luminous indicator.

In one embodiment, the dynamic visual stimulation system comprises a display screen to be disposed, when the device is mounted, in line of sight to render a dynamic visual stimulus displaceable to stimulate the oculomotor response.

In one embodiment, the display screen comprises a widescreen display to be disposed, when the device is mounted, in direct unrefracted line of sight to render a horizontally displaceable stimulus in a wide binocular field of view to stimulate a wide field of view oculomotor response thereto.

In one embodiment, the dynamic stimulation system comprises a set of light sources mountable on the user's head to present, in operation, a visual stimulus at a corresponding plurality of physical locations at respective relative distances to the user's eyes.

In one embodiment, the head-mountable device further comprises a wireless communication interface for digitally relaying data representative of the oculomotor response to a remote computation device.

In accordance with another aspect, there is provided a system for remotely performing an oculomotor assessment of a user, the system comprising: a digital operator application remotely executable on a remote computing device having a communication interface and executable to process oculomotor assessment data, a head-mountable device comprising a housing defining an immersive internal visual stimulation chamber therein, the housing further comprising an external assessment indicator for local output, a communication interface, a dynamic visual stimulation system operatively disposed to operate within the immersive internal visual stimulation chamber to render a dynamic visual stimulus to stimulate an oculomotor response thereto, an eye tracking system configured to monitor the oculomotor response, and a digital data processor. The digital data processor is operable to execute digital instructions for performing the oculomotor assessment via the dynamic visual stimulation system, digitally relay the oculomotor assessment data representative of the oculomotor response to the digital operator application to screen for an oculomotor-related health risk based at least on the oculomotor response thereto as monitored via the eye tracking system, and output, via the external assessment indicator, a screening indicator representative of the health risk.

In one embodiment, the external assessment indicator is physically disposed so to be locally perceivable by an individual facing the user upon which the device is mounted.

In one embodiment, the external assessment indicator comprises a colour-coded luminous indicator.

In one embodiment, the dynamic visual stimulation system comprises a display screen to be disposed, when the device is mounted, in line of sight to render a dynamic visual stimulus displaceable to stimulate the oculomotor response.

In one embodiment, the digital operator application is further executable to render a graphical user interface (GIU) and receive as input therefrom manual digital control of the dynamic visual stimulus such that a stimulus displacement on the display screen corresponds with a manual displacement entered via the GUI.

In accordance with another aspect, there is provided a system for performing an oculomotor assessment of a user, the system comprising: a digital operator application executable on a computing device having a communication interface; a head-mountable device comprising a housing defining an immersive internal visual stimulation chamber therein, a communication interface to the computing device, a display screen operable within the immersive internal visual stimulation chamber to be disposed, when the device is mounted, in line of sight to render a dynamic visual stimulus displaceable to stimulate an oculomotor response thereto, and an eye tracking system configured to monitor the oculomotor response, and a digital data processor operable to execute digital instructions for performing the oculomotor assessment via the display screen. The digital operator application is executable to render a graphical user interface (GIU) on the computing device and receive as input therefrom manual digital control of the dynamic visual stimulus such that a stimulus displacement on the display screen corresponds with a manual displacement entered via the GUI.

In one embodiment, the display screen is disposed, when the device is mounted, in direct unrefracted line of sight to render a horizontally displaceable visual stimulus in a wide binocular field of view to stimulate a wide field of view oculomotor response thereto.

In accordance with another aspect, there is provided a system for performing an oculomotor assessment of a user, the system comprising: a dynamic visual stimulus; an actuator operable to displace the dynamic visual stimulus; an eye tracking system configured to monitor an oculomotor response of the user to the dynamic visual stimulus; and a digital data processor in communication with the actuator and the eye tracking system and operable to execute digital instructions for performing the oculomotor assessment. The oculomotor assessment is performed by activating the actuator to present the dynamic visual stimulus to the user in accordance with the oculomotor assessment, recording the oculomotor response, and outputting a signal representative of the oculomotor response.

In one embodiment, the dynamic visual stimulus comprises a light source.

In one embodiment, the system further comprises a display screen operable by the digital data processor to render visual content thereby, wherein the dynamic visual stimulus comprises the visual content.

In one embodiment, the visual content comprises a target stimulus rendered to be perceived by the user as moving in one or more dimensions in a plane characterising the display screen.

In one embodiment, the visual content comprises a variably sized stimulus rendered to be perceived as moving towards or away from the user.

In one embodiment, the dynamic visual stimulus comprises a light emitting diode.

In one embodiment, the actuator is operable to displace the dynamic visual

stimulus in a first dimension towards or away from the user.

In one embodiment, the actuator is operable to displace the dynamic visual stimulus in a second dimension.

In one embodiment, the actuator is operable to displace the dynamic visual stimulus in a third dimension.

In one embodiment, the oculomotor assessment comprises a vergence assessment.

In accordance with another aspect, there is provided a method for performing an ocular cognitive impairment assessment of a user, the method executed using a digital data processor in communication with each of an actuator operable to displace a dynamic visual stimulus and an eye tracking system configured to monitor an oculomotor response to the dynamic visual stimulus, the method comprising: activating the actuator to present the dynamic visual stimulus at multiple distinct physical locations relative to the user in accordance with the ocular cognitive impairment assessment; recording the oculomotor response of the user indicative of a risk of cognitive impairment; and outputting a signal representative of the oculomotor response.

In accordance with another aspect, there is provided a cognitive impairment assessment device for performing a vision-based cognitive impairment assessment, the cognitive impairment assessment device comprising: a user-interfacing portion to perform the vision-based cognitive impairment assessment therethrough in alignment with the user's eyes, and a load-bearing portion structurally coupled with the user-interfacing portion and housing at least some hardware operable to implement the vision-based cognitive impairment assessment via the user-interfacing portion, so to at least partially transfer a weight of the cognitive impairment assessment device thereto.

In one embodiment, the device comprises a display configured to render visual content perceptible via the user-interfacing portion, and a digital data processor operable to execute digital instructions for performing the vision-based cognitive impairment assessment. The assessment comprises rendering a visual stimulus to the user via the display, recording a physiological response to the visual stimulus indicative of a risk of cognitive impairment, and outputting a signal representative of the risk.

In one embodiment, the device comprises a light field display configured to render visual content perceptible via the user-interfacing portion as originating from distinct depths, and a digital data processor operable to execute digital instructions for performing the vision-based cognitive impairment assessment. The cognitive impairment assessment comprises rendering a visual stimulus to the user via the light field display, wherein the visual stimulus comprises visual content rendered to be perceived as originating from a plurality of designated depths, recording a physiological response to the visual stimulus indicative of a risk of cognitive impairment, and outputting a signal representative of the risk.

In one embodiment, an interface between the load-bearing portion and the user-interfacing portion comprises a rotatable junction so to allow a relative motion between the user-interfacing portion and the load-bearing portion.

In one embodiment, the device comprises a sensing element operable to acquire rotation data related to the relative motion.

In one embodiment, the vision-based cognitive impairment assessment comprises assessing a risk of cognitive impairment based at least in part on the rotation data.

In one embodiment, the device further comprises one or more motion sensors operable to acquire motion-related data.

In one embodiment, the one or more motion sensors are disposed on one or more of the user-interfacing portion or the load-bearing portion.

In one embodiment, the vision-based cognitive impairment assessment comprises assessing a risk of cognitive impairment based at least in part on the motion-related data.

In one embodiment, the load-bearing portion comprises a load-bearing handle or tripod.

In one embodiment, the load-bearing portion comprises a user shoulder mount.

In one embodiment, an interface between the load-bearing portion and the user-interfacing portion comprises a curved or semi-spherical interface to allow a relative translation of the user-interfacing portion and the load-bearing portion.

In one embodiment, an interface between the load-bearing portion and the user-interfacing portion comprises a registration point to favour a designated configuration of the user-interfacing portion relative to the load-bearing portion.

In one embodiment, an interface between the load-bearing portion and the user-interfacing portion comprises a restriction so to limit a range of motion of the user-interfacing portion relative to the load-bearing portion.

In one embodiment, an interface between the load-bearing portion and the user-interfacing portion comprises a flexible material coupling the user-interfacing portion with the load-bearing portion while allowing a relative motion therebetween.

In one embodiment, the device comprises a force sensor configured to acquire data related to an attempted motion of the user-interfacing portion relative to the load-bearing portion.

In one embodiment, the user-interfacing portion comprises a head-mountable portion displaceable relative to the load-bearing portion in response to head motion.

In one embodiment, the head-mountable portion comprises at least some complementary hardware operatively coupled to the load-bearing portion.

In accordance with another aspect, there is provided a cognitive impairment assessment device for assessing a cognitive impairment of interest in a user, the cognitive impairment assessment device comprising: a display configured to render a visual stimulus in accordance with a cognitive impairment assessment; an eye tracking system configured to monitor an oculomotor response to the visual stimulus; a user-interfacing portion configured to interface with a user and comprising a sensor operable to detect a user head motion; and a digital data processor in communication with the display, the eye tracking system, and the sensor, and operable to execute digital instructions for performing the cognitive impairment assessment. The cognitive impairment assessment comprises rendering a visual stimulus to the user via the display, recording a physiological response to the visual stimulus indicative of a risk of cognitive impairment, the physiological response comprising data related to the oculomotor response and the user head motion, and outputting a signal representative of the risk.

In one embodiment, the display comprises a light field display configured to render a visual stimulus perceptible as originating from distinct depths.

In one embodiment, the visual stimulus is rendered to be perceived as moving between the distinct depths.

In one embodiment, the cognitive impairment assessment comprises a Near Point of Accommodation (NPA) assessment.

In one embodiment, the cognitive impairment assessment comprises a vergence-related cognitive impairment assessment.

In one embodiment, the cognitive impairment assessment device comprises an onboard digital data storage device having the digital instructions stored thereon.

In one embodiment, the cognitive impairment assessment device comprises a network communication device configured to communicate wirelessly over a network.

In one embodiment, the digital data processor is further operable, via the network communication device, to update the digital instructions in accordance with updated digital instructions accessible on a remote device.

In one embodiment, the device is remotely operable over the network so to perform a remote telemedicine assessment via a remote medical specialist.

In one embodiment, the cognitive impairment assessment system is portable.

In one embodiment, the cognitive impairment assessment device further comprises a portable power source for powering the cognitive impairment assessment device.

In one embodiment, the cognitive impairment assessment is selectable form a plurality of cognitive impairment assessment profiles each comprising a different set of cognitive assessment tests.

In one embodiment, at least some of the profiles correspond to a designated user activity at risk of subjecting the user to a cognitive impairment incident.

In one embodiment, the cognitive impairment assessment device further comprises a reference sensor configured to provide reference data related to the sensor operable to detect a user head motion.

In one embodiment, the user-interfacing portion comprises a lightweight head-mounting interface.

In one embodiment, the sensor is in wireless communication with the digital data processor.

In one embodiment, the digital instructions further comprise a calibration process to establish a baseline head position, and wherein the user head motion is monitored with respect to the baseline head position.

The cognitive impairment assessment device of any one of Claims 90 to 106, wherein the digital instructions further comprise instructions to decouple the user head motion from the oculomotor response.

Other aspects, features and/or advantages will become more apparent upon reading of the following non-restrictive description of specific embodiments thereof, given by way of example only with reference to the accompanying drawings.

### BRIEF DESCRIPTION OF THE FIGURES

Several embodiments of the present disclosure will be provided, by way of examples only, with reference to the appended drawings, wherein:

FIGS. 1A to 1E are images of various perspective views of the exterior of an exemplary head-mountable device (HMD) for performing oculomotor assessments,

FIGS. 1F to 1J are images of various perspective views of exemplary internal components of the HMD of FIGS. 1A to 1E, and FIGS. 1K to 1M are images of various side and top views illustrating an exemplary viewing screen configuration within the HMD of FIGS. 1A to 1E, in accordance with various embodiments;

FIGS. 2A, 2B, 2C and 2D are perspective, exploded perspective, side and exploded side view schematics, respectively, of an alternative exemplary HMD configuration comprising load-bearing and user-interfacing portions, in accordance with one embodiment;

FIGS. 2E, and 2F to 2H are external perspective, and partially assembled internal perspective views of another alternative HMD configuration comprising respective light field display screens for performing oculomotor assessments, in accordance with another embodiment;

FIG. 3A is a screenshot of an exemplary graphical user interface (GUI) operable to provide user-control over oculomotor assessment parameters and to monitor and visualise metrics associated with an oculomotor assessment, in accordance with one embodiment; FIG. 3B is a screenshot of an exemplary GUI for providing control over an oculomotor assessment, in accordance with one embodiment; and FIGS. 3C to 3F are screenshots of exemplary regions of the GUI of FIG. 3A for monitoring various metrics associated with an oculomotor assessment, in accordance with various embodiments;

FIGS. 4A and 4B are screenshots of an exemplary GUI illustrating different exemplary saccadic assessments, in accordance with different embodiments;

FIGS. 5A and 5B are screenshots of an exemplary GUI illustrating different exemplary smooth pursuit assessments; and FIG. 5C is a screenshot of an exemplary GUI illustrating the displacement of a target stimulus and user gaze during an exemplary smooth pursuit assessment, in accordance with different embodiments;

FIG. 6 is a schematic illustrating an exemplary reaction time assessment, in accordance with one embodiment;

FIG. 7 is a schematic illustrating an exemplary subjective visual assessment that may be performed using an HMD, in accordance with one embodiment;

FIGS. 8A and 8B are schematics illustrating exemplary optokinetic nystagmus (OKN) assessments that may be performed using an HMD, in accordance with some embodiments;

FIGS. 9A and 9B are schematics illustrating exemplary GUIs for controlling and monitoring an exemplary vergence assessment; and FIGS. 9C to 9E are schematics illustrating various general aspects of a vergence assessment, in accordance with some embodiments;

FIGS. 10A to 13B are schematics illustrating various exemplary configurations of visual stimuli to perform various exemplary oculomotor assessments using various exemplary HMD configurations, in accordance with various embodiments;

FIGS. 14 and 15 are schematics illustrating exemplary connectivity between various exemplary components of exemplary HMDs configured for wired and wireless connectivity, respectively, to external devices, in accordance with some embodiments; and

FIGS. 16 and 17 are schematics illustrating exemplary assessment processes that may be performed using various exemplary HMD systems, in accordance with various embodiments.

Elements in the several figures are illustrated for simplicity and clarity and have not necessarily been drawn to scale. For example, the dimensions of some of the elements in the figures may be emphasized relative to other elements for facilitating understanding of the various presently disclosed embodiments. Also, common, but well-understood elements that are useful or necessary in commercially feasible embodiments are often not depicted in order to facilitate a less obstructed view of these various embodiments of the present disclosure.

### DETAILED DESCRIPTION

Various implementations and aspects of the specification will be described with reference to details discussed below. The following description and drawings are illustrative of the specification and are not to be construed as limiting the specification. Numerous specific details are described to provide a thorough understanding of various implementations of the present specification. However, in certain instances, well-known or conventional details are not described in order to provide a concise discussion of implementations of the present specification.

Various apparatuses and processes will be described below to provide examples of implementations of the system disclosed herein. No implementation described below limits any claimed implementation and any claimed implementations may cover processes or apparatuses that differ from those described below. The claimed implementations are not limited to apparatuses or processes having all of the features of any one apparatus or process described below or to features common to multiple or all of the apparatuses or processes described below. It is possible that an apparatus or process described below is not an implementation of any claimed subject matter.

Furthermore, numerous specific details are set forth in order to provide a thorough understanding of the implementations described herein. However, it will be understood by those skilled in the relevant arts that the implementations described herein may be practiced without these specific details. In other instances, well-known methods, procedures and components have not been described in detail so as not to obscure the implementations described herein.

In this specification, elements may be described as ‘configured to’ perform one or more functions or ‘configured for’ such functions. In general, an element that is configured to perform or configured for performing a function is enabled to perform the function, or is suitable for performing the function, or is adapted to perform the function, or is operable to perform the function, or is otherwise capable of performing the function.

It is understood that for the purpose of this specification, language of ‘at least one of X, Y, and Z’ and ‘one or more of X, Y and Z’ may be construed as X only, Y only, Z only, or any combination of two or more items X, Y, and Z (e.g., XYZ, XY, YZ, ZZ, and the like). Similar logic may be applied for two or more items in any occurrence of ‘at least one . . . ’ and ‘one or more . . . ’ language.

Unless defined otherwise, all technical and scientific terms used herein have the same meaning as commonly understood by one of ordinary skill in the art to which this invention belongs.

Throughout the specification and claims, the following terms take the meanings explicitly associated herein, unless the context clearly dictates otherwise. The phrase ‘in one of the embodiments’ or ‘in at least one of the various embodiments’ as used herein does not necessarily refer to the same embodiment, though it may. Furthermore, the phrase ‘in another embodiment’ or ‘in some embodiments’ as used herein does not necessarily refer to a different embodiment, although it may. Thus, as described below, various embodiments may be readily combined, without departing from the scope or spirit of the innovations disclosed herein.

In addition, as used herein, the term ‘or’ is an inclusive ‘or’ operator, and is equivalent to the term ‘and/or,’ unless the context clearly dictates otherwise. The term ‘based on’ is not exclusive and allows for being based on additional factors not described, unless the context clearly dictates otherwise. In addition, throughout the specification, the meaning of ‘a,’ ‘an,’ and ‘the’ include plural references. The meaning of ‘in’ includes ‘in’ and ‘on.’

The term ‘comprising’ as used herein will be understood to mean that the list following is non-exhaustive and may or may not include any other additional suitable items, for example one or more further feature(s), component(s) and/or element(s) as appropriate.

While it was once assumed that the hallmark of a concussion was a loss of consciousness, recent evidence suggests that a majority of people diagnosed with a concussion did not actually experience this symptom. Rather, diagnosis of a traumatic brain injury (TBI) is notoriously challenging, in part due to variability of associated symptoms and the severity thereof. For instance, measurements of TBI severity are typically assessed using a CT structural imaging scan, and/or assessment of the level of consciousness of a patient and the duration of post-traumatic amnesia. Evaluation of severity may then be assessed on a number of scales, such as the Glasgow coma score (CGS). Further, a concussion is a form of TBI that may be considered a functional, rather than a structural injury. In some cases, tissue damage resulting from a jolt to the head may bruise blood vessels, resulting in tissue damage and chemical variations that may degrade information processing throughout the brain, which ultimately may affect sensorimotor functions.

Recent evidence further suggests that oculomotor behaviour may serve as a biomarker in the assessment of a potential TBI. For instance, up to 80% of concussed athletes show some eye dysfunction. The oculomotor system being a relatively accessible portion of the nervous system, assessment of eye function may thus provide valuable information in the evaluation of the presence or degree of cognitive impairment. For example, after a concussion, common ensuing visual disorders may include convergence insufficiency (CI), accommodative insufficiency (AI), and mild saccadic dysfunction (SD). Since a mild concussion is frequently associated with abnormalities of saccades, pursuit eye movements, convergence, accommodation, and the vestibular-ocular reflex, evaluating the vision system of an individual suspected of being cognitively impaired with respect to one or more of these aspects may prove useful in the early diagnosis and/or categorisation of a TBI. Further, such assessment may not only relate to the assessment of a concussion or post-concussion syndrome (PCS), but also to a host of other cognitive impairments, such as autism, PTDS, or schizophrenia, to name a few.

Oculomotor behaviour is typically categorised based in part on the relative amounts of activity observed in respective portions of the brain. For instance, fixations typically involve maintaining eye position in a relatively still state in order to hold the image of a stationary target on the fovea, giving rise to a high degree of visual acuity. Smooth pursuits, on the other hand, relate to the tracking of a moving stimulus to stabilise an image on the fovea. These may be considered a two-phase process, wherein initiation relates to the movement of the eye while no information is recorded, and maintenance relates to the recording of an internal representation of the target in motion as the brain updates and enhances performance of the pursuit of the moving target. Of particular interest due to their relationship with cognitive and motor processes, another category of oculomotor behaviour comprises saccades, which relate to the rapid movement of the fovea between two fixation points, and are often characterised by a velocity, duration, accuracy, and initiation time.

Accordingly, self-paced saccadic eye movements, for instance, have been designated as a potential biomarker for some brain-related injuries, such as post-concussion syndrome, and may be monitored to assess a recovery status associated therewith. Further, various multidimensional methods have been proposed to detect and characterise sensorimotor deficits associated with TBI. For instance, there has been demonstrated a link between higher order visual perception/cognition and eye movements, which may be related to impairment and/or reduction in accuracy, precision, and speed of information processing within cortical circuits. To name one example, Liston et al. (Liston D B, Wong L R. Stone L S., ‘Oculometric Assessment of Sensorimotor Impairment Associated with TBI’, Optom Vis Sci. 2017; 94(1):51-59) found that some individuals having experienced a TBI reported a degradation of oculometrics, such as pursuit latency, initial pursuit acceleration, pursuit gain, catch-up saccade amplitude, proportion smooth tracking, or speed responsiveness. In another example, Hunfalvay et al. (Hunfalvay, M, et al., ‘Horizontal and Vertical Self-Paced Saccades as a Diagnostic Marker of Traumatic Brain Injury’, Concussion 2019; 4(2):CNC60) established eye tracking tests to measure horizontal and vertical saccades as a proxy for neural deficits, finding that individuals with a concussion were correctly identified 77% and 64% of the time, respectively, while similar results were achieved for the identification of individuals without a concussion.

Digital eye tracking tests such as Hunfalway, et al., as well as other similar approaches, may offer a degree of precision and analysis that comprise an improvement over conventional ‘follow my finger’ tests performed by a neurologist or neuro-optometrist. For instance, the FDA has approved the RightEye™ eye tracking system as a means of recording and analysing eye movements as a patient tracks stimuli displayed on a 2D screen to support identification of visual tracking impairments. Similarly, the FDA-approved EyeBOX® by Oculogica®, and EYE-SYNC® by SyncThink™, track eye movements as a patient follows objects on a display screen to assist in the assessment of TBI, the latter providing a head-mounted display connected to a computer tablet. While such systems may provide the ability to perform some oculomotor tests related to the assessment of potential cognitive impairments, such as concussions, with a relatively high degree of accuracy, patients are typically restricted to tracking objects at a fixed distance in two dimensions. Various other important oculomotor assessments, however, such as those related to convergence, may require a depth component, wherein the object to be tracked or focused on changes depth planes, or moves towards or away from the eyes of the subject. While such assessments have been contemplated using a head-mounted display, for instance in U.S. patent application Ser. No. 19/0,082,954 entitled ‘Objective Testing of Vergence Dysfunction for Diagnosis and Vergence Recovery Convalescence Using Dynamic Vergence Testing Platform Including 3D Head Mounted Display System with Integrated Eye Tracking Technology’ published Mar. 21, 2019 to Kiderman and Ashmore, such systems continue to rely on 2D displays that attempt to trick the visual system of the user into perceiving a change in object depth by presenting stimuli to be tracked in the context of background stimuli.

Taking this notion one step further, U.S. Pat. No. 10,719,992 entitled ‘Augmented Reality Display System for Evaluation and Modification of Neurological Conditions, Including Visual Processing and Perception Conditions’ and issued to Samec, et al. on Jul. 21, 2020 further attempts to mimic the effects of light originating from an object at a given depth, while attempting to improve the accommodation-vergence reflex, by manipulating the divergence properties of light emanating from waveguides in augmented reality display goggles that transmit light from an external environment. Such AR systems, however, are not ideal for cognitive impairment testing. For instance, various tests benefit from an isolated viewing environment that is, for instance, controlled, and/or free of distraction, which otherwise may influence eye movement, or assessment thereof. Further, the manipulation of the divergence properties of light from waveguides is not particularly well suited to providing a range of perceived depth planes, or a sufficient quality of displayed content (e.g. resolution), to adequately perform a cognitive assessment.

Light field displays, on the other hand, offer the ability to directly control the image plane or depth at which content is virtually located and/or perceived. Accordingly, a light field display system equipped with, for instance, eye tracking functionality, may enable assessment of oculomotor behaviour, not only as it pertains to the performance of two-dimensional tests, as described above, but also to three-dimensional assessments that may be more ‘true-to-life’ than those provided from static 2D or augmented reality display systems that rely on tricking the visual system with depth cues. For example, Applicant's co-pending U.S. Patent Application No. 63/179,057 entitled ‘Cognitive Impairment Testing System, and Method Using Same’, the entire contents of which are hereby incorporated by reference, discloses light field-based systems and methods operable to perform a wide range of cognitive impairment assessments, including both 2D and 3D tests for evaluating the oculomotor system of a user. While such light field-based systems and methods offer a means for performing various ocular assessments, such as vergence, saccadic, and pursuit tests, the ability to create a light field requires, in addition to a 2D display screen, various optical components, such as lenses, microlens arrays or like light field shaping elements, and significant processing resources to render ray-traced content. Such components generally add weight and complexity, increase costs, and reduce transportability of head-mounted devices.

Conversely, some of the systems and methods described herein provide, in accordance with different embodiments, different examples for presenting, while user gaze is monitored and within a single head-mounted device, various stimuli disposed or moving in up to three dimensions, without requiring the generation of a light field (e.g. using unrefracted light). Accordingly, various systems or devices as herein described may provide stimuli for various 1D, 2D, or 3D assessments and/or exercises with fewer optical components and processing resources, and less circuitry than is required for conventional light field display systems. However, it will be appreciated that various embodiments herein described may additionally or alternatively provide for a system or method employing a head-mounted display for performing light field-based assessments while providing improvements over conventional light field systems. For instance, various embodiments provide solutions to challenges associated the inherent complexity, bulk, weight, and lack of transportability of light field systems, and provide improved dynamic range as compared to traditional light field systems with respect to the presentation of visual content, and the range and ultimate performance of assessments.

In accordance with various embodiments, an assessment system or device, or a method using same, as herein described, may comprise a head-mounted device or head-mountable device (HMD) that may provide a medical practitioner with quantitative metrics pertaining to eye and head dynamics as the wearer of the HMD (the ‘user’) is directed to follow with their eye(s) a stimulus or stimuli presented in accordance with a designated pattern or sequence. In accordance with some embodiments, such a pattern or sequence may correspond with a 1D, 2D, or 3D oculomotor assessment or exercise. Such data may then be used by the practitioner for subsequent analysis to, for instance, inform decisions or practices with respect to the user.

For example, and without limitation, an HMD as herein described may be employed to screen for or assess a variety of cognitive functions or conditions, non-limiting examples of which may include TBI, attention deficit hyperactivity disease, Alzheimer's disease, Parkinson's disease, Tourette's syndrome, progressive supranuclear palsy, or motor neuron disease. As such, and for simplicity, various embodiments of the systems and methods described herein may be referred to as ‘assessment systems’ or ‘assessment methods’; however, it will be appreciated that various embodiments may relate to a system or method for various other applications, non-limiting examples of which may include lie detection, or oculomotor stimulation for, for example, brain injury or post-surgical rehabilitations, or cognitive training, without departing from the general scope or nature of the disclosure. Accordingly, it will be appreciated that an ‘oculomotor assessment’, an ‘assessment’, a ‘cognitive assessment’, or a ‘visual test’ or ‘visual assessment’, which may be referred to interchangeably herein, may additionally or alternatively relate to the monitoring of a user response to stimuli presented in accordance with an oculomotor exercise, training regime, or the like.

In accordance with various embodiments, various eye tracking systems or methods currently or as yet to be known in the art may be employed in within an HMD to record, monitor, and/or analyse, for instance, pupil size, position, movement, or the like, before, during, or after an oculomotor assessment. For instance, and without limitation, various eye tracking systems may relate to those employing visible or IR cameras to track pupils during vision-based, caloric, oculomotor, vestibular, or reaction time (OVRT) assessments. Similarly, and as will be further described below, an HMD may comprise one or more motion sensors, gyroscopes, accelerometers, or the like, to track user and/or head motion, or a relative motion, before, during, or after and assessment or exercise. Such data may be communicated to a practitioner to, for instance, inform assessments, therapy practices, or the like. That is, while some embodiments relate to direct processing of assessment data on-board an HMD or via a processing resource coupled therewith to filter, calculate, or infer various metrics related to oculomotor behaviour and/or function, various embodiments may additionally or alternatively relay data or inferred metrics for professional human analysis, depending on, for instance, the particular application, assessment, or metric at issue.

In accordance with various embodiments, a visual stimulus may be presented within an assessment system such that it may be perceived by a user as being disposed at a one or more designated locations, or moving in up to three dimensions, in accordance with various oculomotor assessments. As will be further described below, a stimulus may be provided in various forms, in accordance with various embodiments. For example, a stimulus or stimuli may comprise a linear, two-dimensional, or three-dimensional array of light sources that are independently addressable and distributed in physical space such that, when different sources are activated or presented as a target of focus for a user, the user attempts to focus in a different spatial regions within the HMD. In some embodiments, this may relate to a pixelated display screen in which individual pixels or groups thereof may be addressed and/or activated to provide a stationary or perceivably moving stimulus in accordance with a designated assessment. For example, a pixelated display screen may activate pixel groups as a stimulus to draw the user's gaze to a particular location in the plane of the screen, whereby the stimulus is digitally moved in the plane of the screen in accordance with an assessment. In accordance with yet other embodiments, such a screen may be further displaced along an axis perpendicular to the plane of the screen to further displace the stimulus in a third dimension. In such embodiments where a stimulus that is physically moving or appears to be moving, the stimulus may also be referred to herein as a ‘dynamic stimulus’.

The following description relates to the use of an HMD applied for the assessment of cognitive function or a cognitive disorder through assessment of the oculomotor system of a user. However, it will be appreciated that such applications are provided for exemplary purposes, only, and that the systems and methods described forthwith may be readily applied in, for instance, therapeutic and/or training applications, without departing from the general scope and nature of the disclosure.

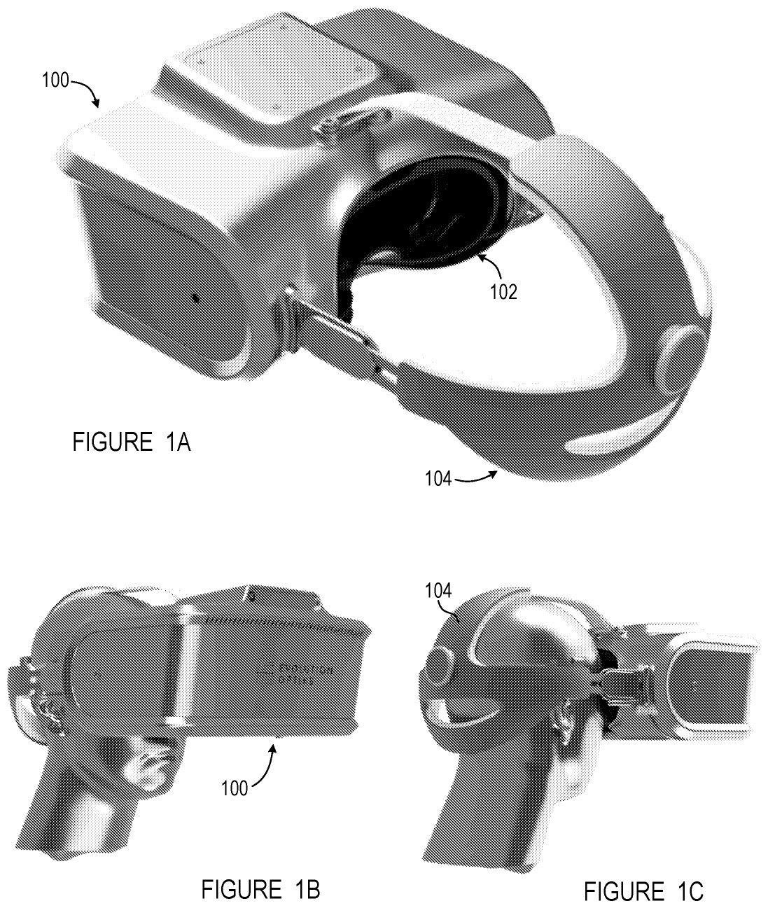

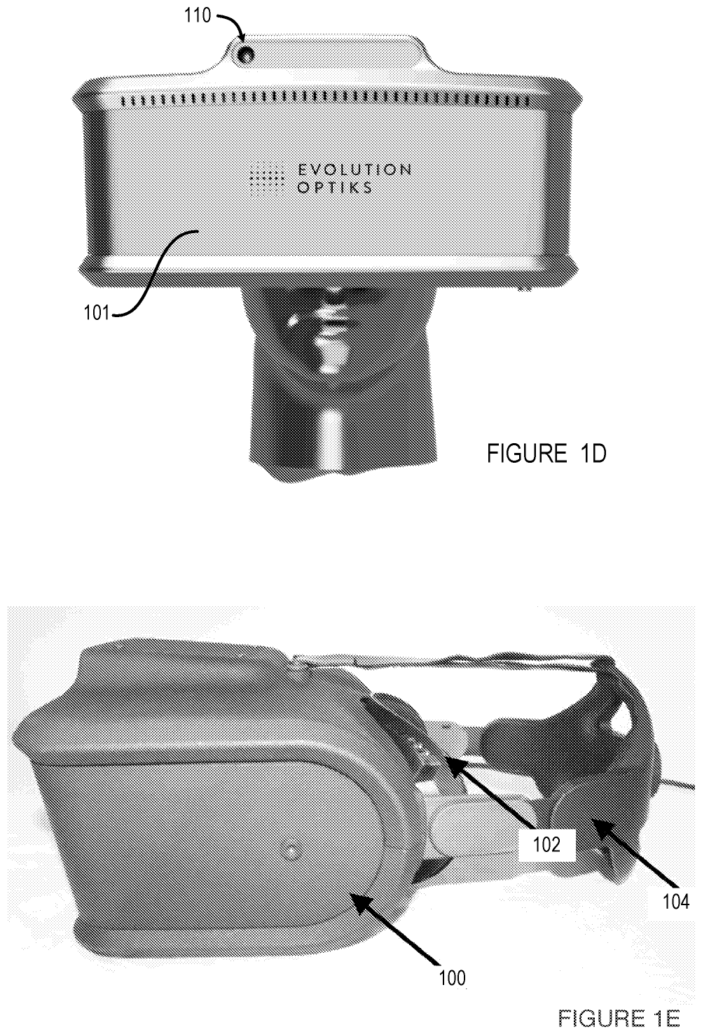

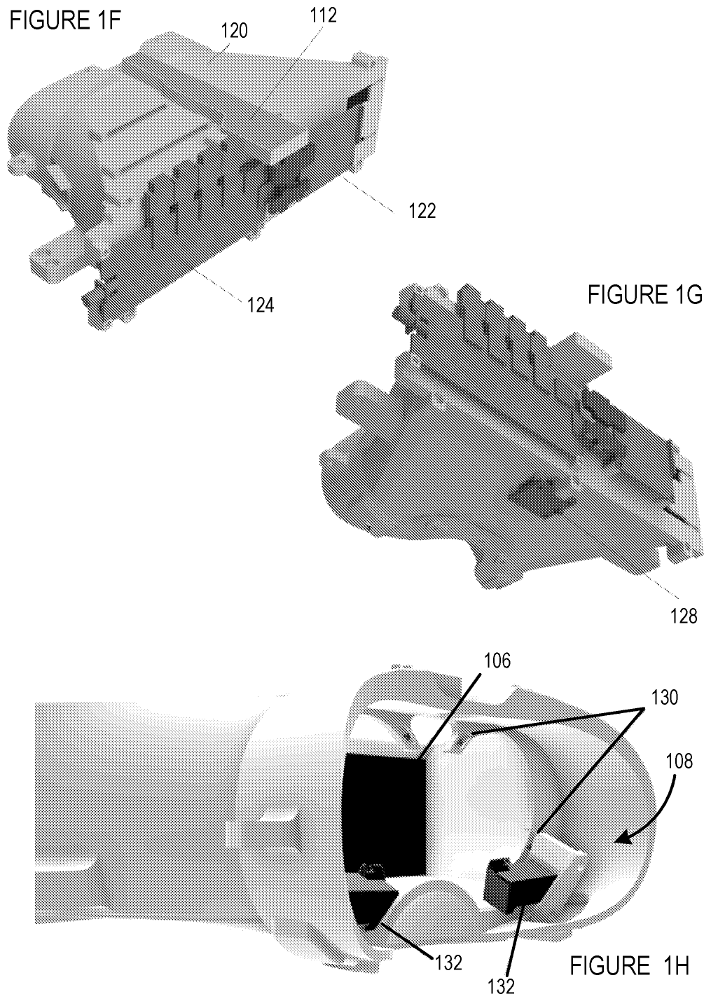

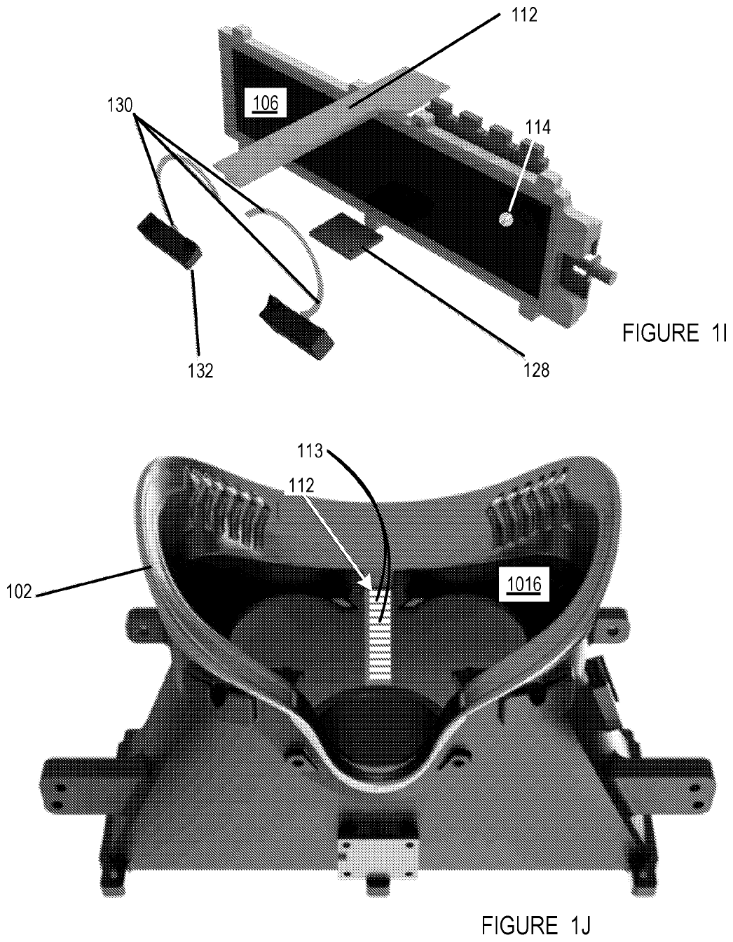

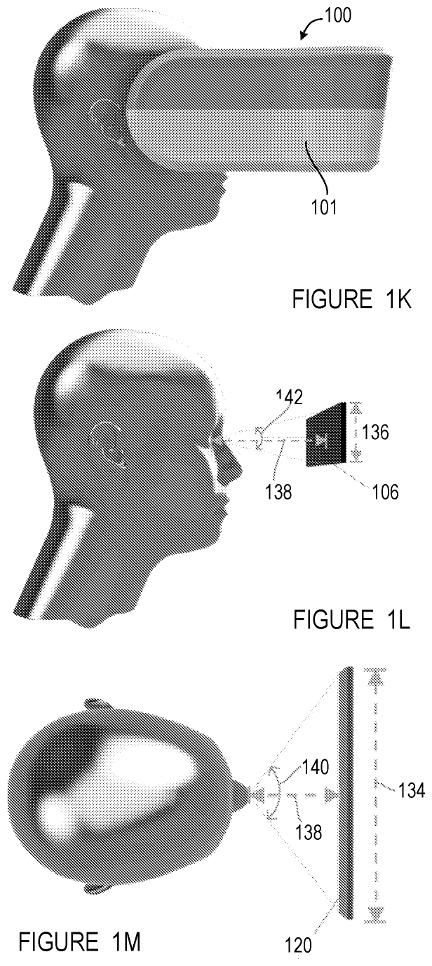

With reference to FIGS. 1A to 1J and in accordance with one embodiment, a head-mountable device (HMD) 100 operable to perform a cognitive impairment assessment and/or screening will now be described. In particular, FIGS. 1A to 1G are exemplary images of the external enclosure 101 of the HMD 100, while FIGS. 1H to 1J are images illustrating an inner configuration of the exemplary HMD 100 for performing an assessment.

In this particular embodiment, the device 100 comprises a face-resting portion 102 that is to be aligned and rested around the user's eyes on their face, and maintained in position via a head-fastening strap or harness 104 that can be adjusted to secure the HMD 100 to the user's head. Various head-mounting, weight-bearing and/or otherwise supportive mechanism, as described herein, may equally be considered herein, as can various mechanical adjustments, interfaces and/or motion-related accommodations may be equally considered within the present context. Similarly, it will be appreciated that the HMD 100, face-resting portion 102, and/or harness 104 may be equipped with various pads or physical structures for user comfort and/or isolation, and/or to provide additional functionality. For example, various embodiments relate to the face-resting portion 102 comprising a padding or flexible component for user comfort and stability during use. In accordance with some embodiments, such a padding may optionally additionally or alternatively serve as a means of blocking external light during performance of one or more assessments, thereby improving user experience and/or the quality of assessments, which may otherwise be hindered by stray light entering the system that may distract the user or contribute to discomfort.

In this particular embodiment, FIGS. 1A to 1E show various digitally rendered perspective views of the HMD 100 while not in use (FIG. 1A), and while mounted to the face of a user (FIGS. 1B to 1D). FIG. 1E is a photograph of the left side of an exemplary HMD 100. Such systems may enable an assessment to be conducted via a visual interface within the HMD 100. In the exemplary embodiment of FIG. 1D, an external indicator 110, such as a colour-coded luminous indicator, is provided in a rearward facing configuration (or indeed, an indicator disposed on the HMD 100 such that it is readily visible to an external non-user of the device) such that a test administrator, or anyone in the presence of the user during performance of the test, can visually obtain an immediate screening result while the user is wearing the device. For example, a red indicator may indicate that a test screening suggests the user is at a relatively high risk of exhibiting cognitive impairment, whereas a green indicator may indicate a lower likelihood of cognitive impairment. Other colour indicators may, for example, reflect that a screening test is underway (i.e. active screening), or again flash or change colours based on a current status or progression of the device. However, unlike a tradition head-mounted device where outputs are constrained to being rendered by the device's internal display, for instance within the context of a virtual reality device or the like, or again, wherein external outputs or results are exclusively made available via a communicatively linked device (e.g. tethered laptop or remote display), the device 100 provides a local operator, administrator and/or assistant (e.g. nurse, doctor, family, friend, bystander, coach, technician, etc.), immediate indication of a screening status in a readily available configuration. Indeed, one assisting a user in the implementation of the test is likely to stand in front of them, thus facing a rearward extent of the device 100, and thus, conveniently positioned to acknowledge an output of the indicator 110. As will be appreciated by the skilled artisan, other types of indicators may be considered without departing from the general scope and nature of the present disclosure. Moreover, various embodiments relate to the use of such an indicator on various other HMD configurations. For instance, a light-field based HMD, and/or an HMD comprise a plurality of parts, as will be further described below, may similarly employ an outward-facing assessment status indicator, without departing from the general scope and nature of the disclosure.

The HMD 100 may further comprise various additional external components, depending on the particular application at hand. For example, and without limitation, the HMD 100 or harness 104 may be equipped with speakers to provide a user of the device with audio content, such as assessment instruction, guidance, and/or feedback before, during, or after an assessment. Such a feature may be of benefit if, for instance, a practitioner is guiding an assessment remotely, or if such guidance is pre-recorded, or computer-generated in real-time in response to assessment progression and/or results. Speakers may further be of value in expanding the range of tests that may be performed with the HMD 100. For instance, while various embodiments relate to the presentation of visual stimuli, other assessments may additionally or alternatively relate to a volume sensitivity in response to an audio stimulus, and/or the ability to perceive and/or translate audio content to performance of an action (e.g. a voluntary oculomotor response within the device, a verbal or physical response, or the like). In accordance with some embodiments, earpieces comprising speakers may be retractable, thereby facilitating transport and deployments, and/or improving the portable, compact, and/or ergonomic nature of the HMD 100.

In accordance with yet other embodiments, an HMD 100 may provide various additional or alternative sensors and/or components for facilitating various other forms of assessments. For instance, earpiece structures may further facilitate, for instance, caloric assessments through the provision of hot or cold fluids and/or various heating elements or other sensors. In accordance with another example, the HMD 100 comprises an ultrasound or like device (e.g. a transcranial Doppler ultrasound device) to evaluate blood flow around the brain of a user. In accordance with some embodiments, such components may be disposed in, on, or in a coupled configuration with the external or outer assembly of the HMD 100, although it will be appreciated that such aspects may be included within the HMD 100, for instance within an external casing 101 of the HMD 100.

Moving within the HMD 100, FIGS. 1F to 1J are digitally rendered images showing various perspective views of exemplary inner components of the HMD 100, in accordance with one exemplary embodiment. In this example, FIG. 1F is a top front right perspective view of an inner enclosing structure 120 configured for coupling to various structural and electronic components of the HMD 100. In this case, the HMD 100 comprises two circuit boards disposed at the font of the device corresponding to a display board 122 and a multiport hub 124 providing connectivity and suitable circuitry for controlling various aspects of stimulus provision and eye position monitoring, as well as facilitating connectivity (e.g. wireless or wired) to external devices. FIG. 1J further shows a vergence testing feature 112, as will be further described below to include a set of distinctly addressable light sources 113, such as LEDs or the like, to be activated in a sequence in accordance with a prescribed vergence test.

In accordance with some embodiments, the HMD 100 comprises one or more position or motion sensors operable to measure, for instance, user movement during assessment. For example, various cognitive impairment assessments known in the art incorporate data related to user head motion. Such data, including, for example, head motion of the subject when asked to track a moving target with their eyes while not moving their head, or a motion pattern observed as a target on which the subject should focus is presented while the user is to rotate their head to one side or the other (or upwards/downwards), may be of avail to a number of assessments. In addition or as an alternative to being sensed as a direct measure of a risk of an impairment (e.g. TBI), and in accordance with some embodiments, such head motion may further be used to provide control data, for instance to normalise data related to oculomotor function, or to correct or eliminate incorrect data for a variety of tests. Accordingly, such data may have standalone value in a cognitive assessment, and/or may serve any one or more of a variety of purposes, in accordance with various embodiments. Various embodiments thus relate to the HMD 100 being operable to acquire user motion data via, for instance, an inertial measurement unit (IMU), gyroscope, accelerometer, or like sensor incorporated therein or thereon. In the exemplary embodiment of FIG. 1G illustrating a bottom front right view of the inner enclosure 120, such a component is shown as an IMU 128 mounted below the inner enclosure 120.

A front right-side perspective view of the inner structures of FIGS. 1F and 1G is shown in FIG. 1H, with the circuit boards 122 and 124 removed for clarity. In this example, the HMD 100 further comprises infrared (IR) light-emitting diodes (LEDs) 130 and embedded cameras 132 as components of a gaze tracking system 108. FIG. 1I further shows these and other components in a top back right perspective view of the device, the inner enclosure casing 120 removed for clarity. In this view, the arcuate shape of the IR source 130 for increased uniformity of eye illumination is seen, in accordance with one embodiment. FIG. 1J is a bottom rear perspective view showing the inner enclosure casing 120, as well as a padding of the face-resting portion 102 and the vergence testing feature 112.

Unlike conventional head-mounted devices as used, for example, in virtual reality applications, the exemplary HMD 100 of FIGS. 1A to 1J provides a user with a direct wide-angled and unrefracted binocular view of a same display screen 106. In doing so, rather than to have each eye gaze focused on respective typically small screens via respective intervening lenses or lens sets, and having a binocular experience virtually produced through rendering software, both eyes can directly view a same wide screen 106, thereby invoking a more natural binocular vision scenario and thus facilitating observation and tracking of a more accurate oculomotor response (i.e. via gaze tracking system 108), resulting in a more accurate cognitive impairment screening. Indeed, in the exemplary HMD 100 of this particular embodiment, the user's immersive viewing experience accommodates a wide horizontal field of view. The provision of an unrefracted wide-angle field of view provides, amongst other advantages, for a greater oculomotor range of motion when conducting different vision-based cognitive impairment tests, such as a wide-angle range for smooth pursuit or Optokinetic Nystagmus (OKN), e.g. including tests such as where a visual stimulus 114 (FIG. 1I) is smoothly tracked across the display 106, or again where a translating pattern of light and dark lines is rendered across the display 106 and a tracking response thereto is monitored. Indeed, for this latter example, a wide-angle field of view is necessary to successfully observe, for instance, a corresponding cognitive impairment, and otherwise generally unachievable using a focused line of sight display.

In this particular embodiment, the display 106 is mounted at the far end of a viewing tunnel 116, or like structure provided in part by the inner enclosure 120, which immerses the user's gaze to the display 106 and blocks out any external stimuli, while allowing for operation of the eye/gaze tracking system 108. Indeed, the minimalist viewing tunnel 116, devoid of any intervening refractive optics, is adapted to minimise any luminous reflections or artefacts and thus, casts the user in a mostly darkened environment where they can focus exclusively on the test stimulus. In some embodiments, an interior surface of the viewing tunnel 116 is provided as an amorphous surface, thereby further reducing internal reflections and visual distractions. For example, the amorphous surface may include, but is not limited to, an amorphous and generally black textured or rugged plastic or like surface, a lined or stretched fabric, opaque stocking, or the like. Accordingly, upon resting the face-resting surface of the portion 102 against the user's face around their eyes, as best viewed in FIG. 1J, a direct, unobstructed and unrefracted wide-angle field of view to the display 106 is provided and confined to the immersive viewing tunnel 116.

The wide-angle field of view of the HMD 100 is schematically illustrated in FIGS. 1K to 1M, where FIG. 1K is a right-side view of the outside of the HMD when in use, and FIG. 1L is the same view as FIG. 1K showing only the display screen 106 of the device relative to the user, while FIG. 1M is a top view of the wide screen 106 of the HMD 100 relative to the user. In this embodiment, the display 106 has a width 134 and height 136, and is disposed at a distance 138 from the eyes of the user when in use, which defines a horizontal viewing angle 140 and vertical viewing angle 142. The dimensions and screen configuration relative to the user may be defined based on, for instance, a particular application at hand (e.g. cognitive impairment assessment, or the like), and/or may be adjustable (e.g. brought nearer to or farther from the user), as will be further described below. In accordance with various embodiments, the horizontal field of view 140 may be within a range of approximately 65 degrees to approximately 120 degrees, and the vertical field of view 142 may range from approximately 15 degrees to approximately 30 degrees. In the exemplary embodiment of FIGS. 1L and 1M, the screen is 106 is approximately 219 mm wide, approximately 55 mm in height, and is disposed approximately 141 mm from the user's eyes, corresponding to a horizontal view angle of approximately 75 degrees and a vertical field of view of approximately 22 degrees, each as measured at a distance when the device is mounted from the center of the user's two eyes to the display. In accordance with some embodiments, such specifications may be selected to achieve a distance between the eye and the display that is comfortable while not being too distant so to produce an excessive moment as a result of any shift in center of gravity of the device.

With reference again to FIG. 1J, the device 100 further comprises, in addition to the display screen 106, a vergence testing feature 112, as will be further described below. Accordingly, an in accordance with various embodiments, the HMD 100 may comprise various components or forms of visual stimuli from which to trigger and monitor an oculomotor response from the user. In this example, the additional visual stimulus in the form of a vergence testing feature 112 comprises a strip of LEDs 113 that can be successively illuminated in guiding the user's gaze toward and away from the display 106 in testing, for example, a near point of convergence. For example, a user manifesting a roughly 4 mm near point of convergence will screen out as likely healthy, whereas one manifesting a near point of convergence greater than 8 mm will screen out as likely exhibiting some signs of cognitive impairment. Any anomalous oculomotor responses may also be detected and observed by the HMD 100.

As further described below, various approaches may be taken to implement a cognitive impairment test via device 100. One or more stored and user or administrator-guiding tests may be executed locally from memory (not shown in FIGS. 1A to 1J), whereby a screening result (for each test or overall) may be output locally via the onboard indicator 110 and/or communicated locally or remotely to a corresponding device and user interface, either way guiding the user or caretaker in deciding whether further care or testing may be required. Similarly, an external interface, either provided via a locally or remotely implemented user interface, may provide greater testing control, such as by providing a suite of automated tests (e.g. preset testing visual patterns and/or sequences), and/or manually adjustable or executable tests, for example, where a user dynamically sets visual stimulus thresholds, boundaries, sizes, speeds, ranges, or the like, or again, manually controls displacement, translation and/or positioning of various visual stimuli. In the later example, for instance, an operator could invoke a touchscreen interface replicating the display 106 such that, via touch control, an operator may directly control a location and displacement of a rendered visual stimulus, thus more closely replicating traditional hand/finger tests performed in the field or in clinic.

As briefly described above, user motion (e.g. head or device motion) may be of value in the assessment of a potential user condition (e.g. a concussion). Moreover, greater value may be extracted from user head motion when the head is less hindered by, for instance, the weight of the HMD 100. For instance, a particularly heavy headset may dampen or otherwise impact head motion, thereby affecting assessment of a potential cognitive impairment. Further, it may not be desirable to encumber or put undue stress on the head or neck of an individual suspected of an injury like a concussion. Accordingly, and in accordance with some embodiments, an HMD may comprise a means for supporting a portion of its weight, such that unnecessary weight is not placed on a patient's head or neck. For example, and in accordance with one embodiment, an assessment device may comprise one or more handles that may be grasped by a hand(s) of the patient or practitioner conducting an assessment, or one or more legs (e.g. a tripod) to support at least a portion of the weight of the system.

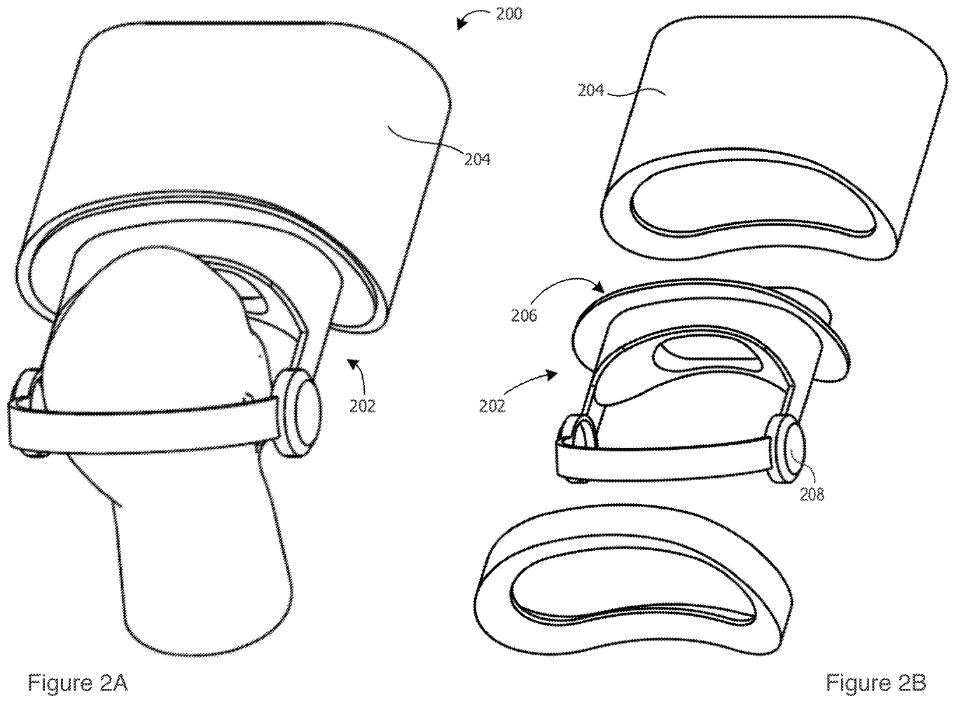

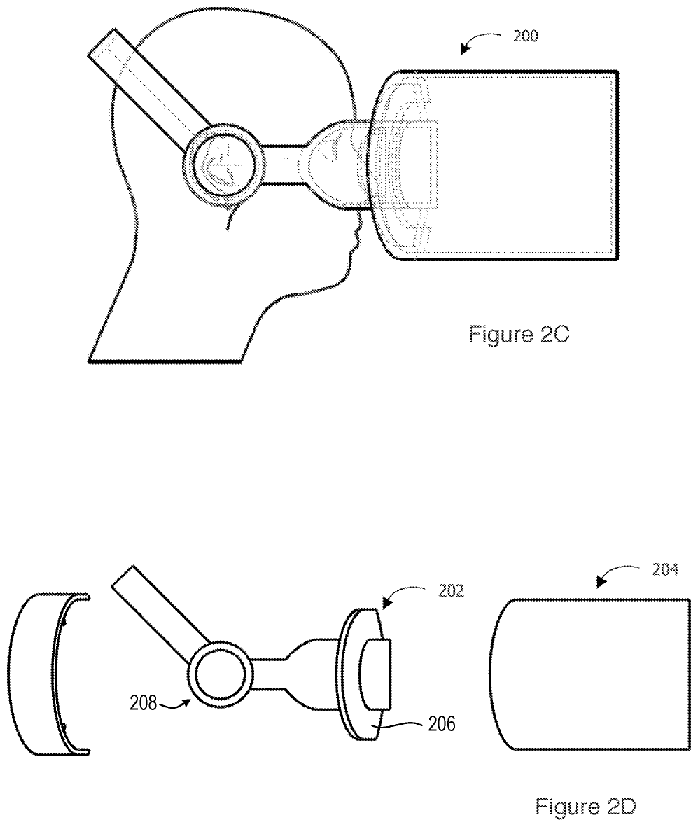

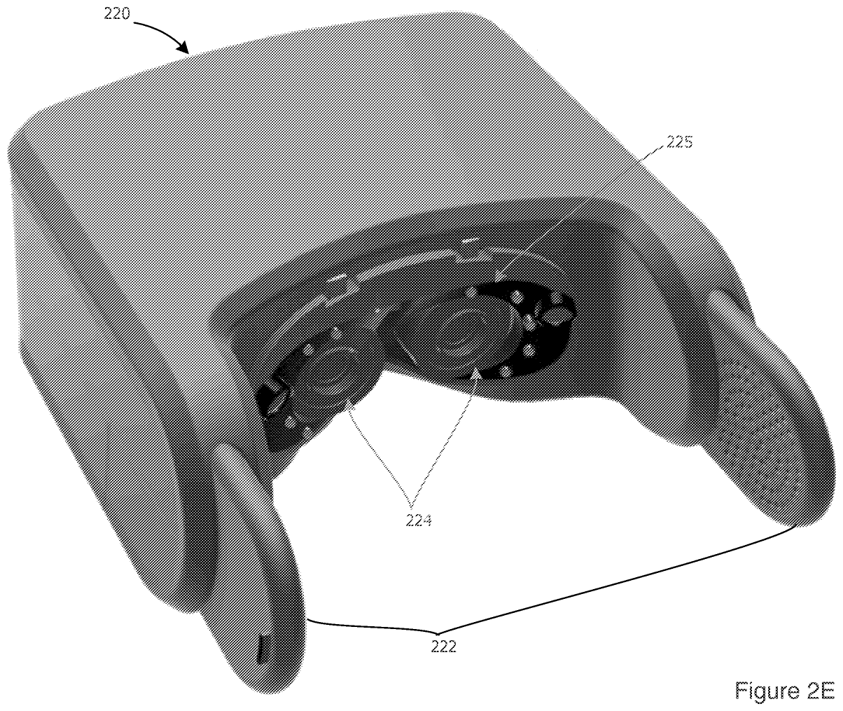

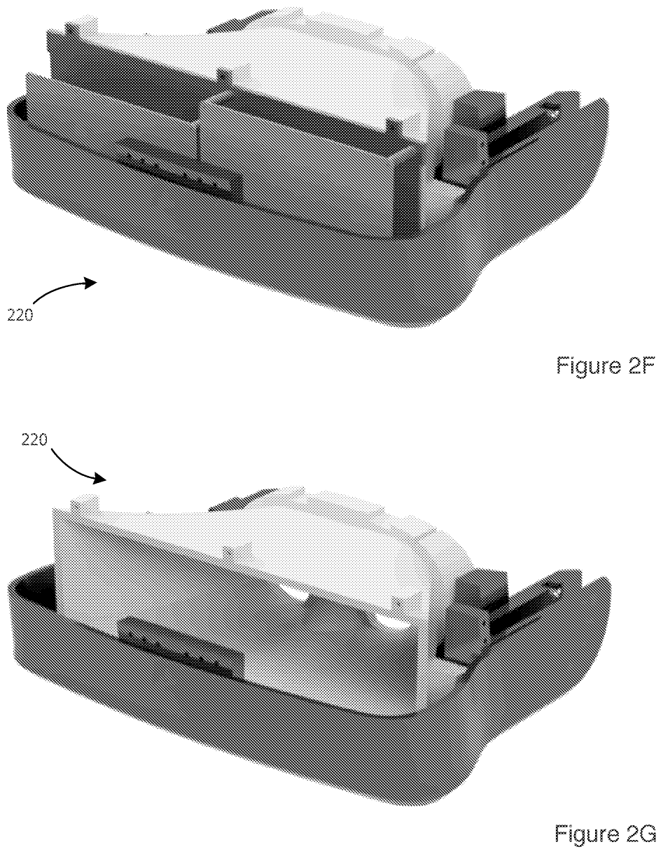

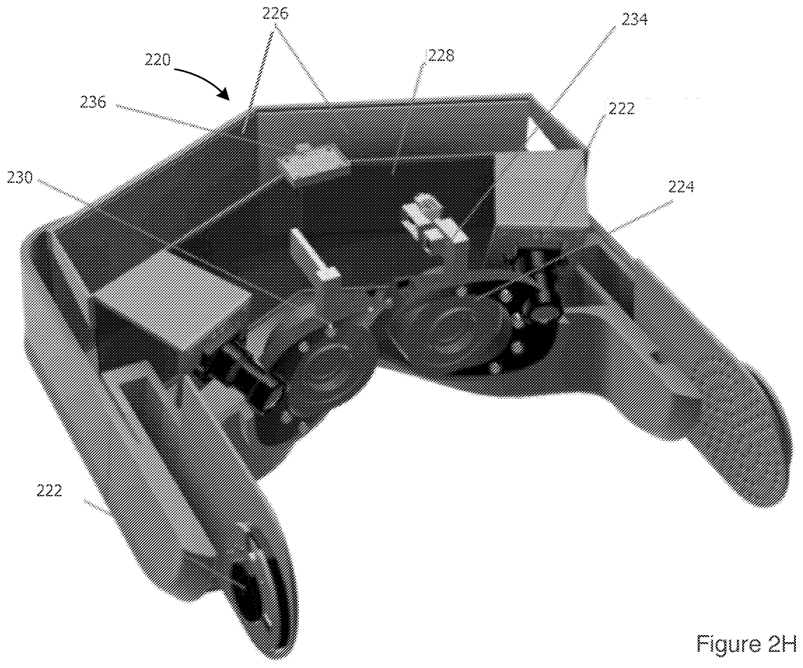

To this end, and in accordance with some embodiments, FIGS. 2A and 2B schematically illustrate an exemplary alternative configuration of an HMD 200 comprising a lightweight, user-interfacing potion 202 and a load-bearing portion 204. In this exemplary embodiment, FIG. 2A shows the device 200 while in use by a subject or user, while FIG. 2B schematically shows an exploded view of various exemplary components of the device 200. In this example, a spherical interface 206 between the lightweight 202 and load-bearing 204 portions enable translation of the lightweight portion 202 relative to the load-bearing portion 204 in any direction as the subject moves their head, although other embodiments relate to an interface that may be restricted in one or more dimensions, comprise one or more registration points or grooves to guide or restrict user motion, and/or comprise one or more sensors (e.g. force sensors).

In this exemplary alternative configuration, the load-bearing portion 204 may comprise, for instance, heavier components related to the provision of various stimuli for an assessment, such as a display screen or other optical components, electronic circuitry, an eye tracking system, or the like. Conversely, while the user-interfacing portion 202 may comprise various components that are beneficially close to or on the patient's head, such as earphones 208, it may, in accordance with various embodiments, otherwise comprise lightweight components or materials such that undue burden is not placed on the user. FIG. 2C further schematically shows a right-side view of the assessment device 200 while in use by a user, while FIG. 2D schematically shows a right-side exploded view of various components of the device 200.

The lightweight portion 202 may, in some embodiments, be rotatable or otherwise be permitted motion relative to the second portion, thereby allowing the subject to move their head relative to the load-bearing portion 204 with relative ease. For example, one or more ball bearings or similar means may allow the lightweight portion 202 to rotate leftward, rightward, upwards, downwards, or a combination thereof relative to the load-bearing portion 204. In accordance with various embodiments, rotation of the components may be monitored using position or like sensors to control for or filter other assessment data, or may serve directly as assessment data. For example, tracking rotation angles between the lightweight and second heavier portions may allow for extraction of the speed, acceleration, or smoothness of head rotation during an assessment.

In some embodiments, such movement may be restricted to within a designated range(s), for instance to ensure that a subject does not overextend or over-rotate in a particular direction(s) (e.g. <5°, <10°, <25°, or the like). For example, and in accordance with one embodiment, a load-bearing portion may comprise two screens, each for displaying content to a respective eye of the subject. The respective viewing regions may be separated or isolated from respective eyes by a divider or other isolating means disposed on one or both of the lightweight portion and load-bearing portion. In such a configuration, the subject may only properly view each screen for an assessment if their head, and thus the lightweight portion of the device interfaced therewith, are relatively rotated by a maximum of, for instance, 10° from the screen normal. In embodiments comprising a single display screen, such as the HMD 100 of FIGS. 1A to 1J having a single wide-angle viewing configuration, it may similarly be preferred to restrict a range of motion of the user so to optimise or limit angular views of the screen. Accordingly, such embodiments may further comprise a means of limiting rotation, such as a groove of a finite length via which relative translation may occur, through structures on one or more of the portions that impede relative motion past a designated angle, or the like.

In some embodiments, the first lightweight and second heavier portions 202 and 204, respectively, may rotate synchronously (i.e. remain in the same relative configuration upon subject motion), for instance via a selectively fixed coupling region therebetween. For example, a system may comprise a translatable junction comprising a ball bearing or other means known in the art for enabling rotation or relative motion between two bodies, while further comprising a latch or other locking mechanism to selectively disengage the translating functionality. For example, relative motion between the lightweight and heavier portions may be locked in cases where such motion is not desired (e.g. during a motionless oculomotor assessment). It will be appreciated that various means may be employed to fix or disengage a locking mechanism to inhibit or allow relative motion between, and that such components are also considered within the scope of the disclosure. For example, a mechanical feature (e.g. a pin or latch) or electromagnetic component may be engaged to fix the relative position of the lightweight and heavier portions when motion is not desired, or released when motion is to be assessed.

In accordance with various embodiments, a multi-part cognitive impairment assessment system comprising a lightweight subject-interfacing portion 202 and one or more load bearing or supporting portions 204 may comprise various interface configurations therebetween to allow for user motion. For instance, and in accordance with one embodiment, FIGS. 2A to 2D schematically show an exemplary configuration of a cognitive impairment assessment device 200 comprising an arcuate or semi-spherical interface 206 between the lightweight, user-interfacing potion 202 and a load-bearing portion 204.

In this example, the spherical interface 206 between the lightweight 202 and load-bearing 204 portions enable translation of the lightweight portion 202 relative to the load-bearing portion 204 in any direction as the subject moves their head. In the exploded view of the device 200 of FIG. 2B, the exemplary interface 206 comprises, on the lightweight subject-interfacing portion 202 and load-bearing portion 204, respectively, a spherically convex surface and a spherically concave surface that, when in use, are in translatable communication such that the lightweight portion 202 may rotate in any direction with respect to the load-bearing portion 204.

In some embodiments, the configuration of the interface 206 between the lightweight portion 202 and the load-bearing portion 204 may comprise a favoured registration point(s) for a preferred orientation(s). For example, it may be preferred for various assessments that the subject faces the screen(s) directly. Accordingly, some embodiments may comprise a registration point(s) or groove(s) on, for instance, surfaces of the interface 206, which encourage the lightweight portion 202 to remain directly perpendicular to a display within the load-bearing portion 204.

In accordance with various embodiments, the impairment assessment device 200, the lightweight 202 and load-bearing portions 204, or an interface 206 thereof may comprise a plurality of such registration points or regions. For example, one embodiment relates to a system having registration points at various relative angles between the lightweight and load-bearing portions so to favour specific orientations (e.g. 0°, and +10° in both or either left/right and up/down directions). In accordance with other embodiments, grooves or other like registration means may favour motion of the lightweight portion 202 to the load-bearing portion 204 in both left/right and up/down directions, for instance to accommodate cognitive assessments in which the user is asked to move their head sideways or up/down while maintaining focus on a fixed point.

In accordance with some embodiments, such registration points may be magnetic, or electromagnetically activated. For instance, the lightweight portion 202 may be loosely held directly in front of (i.e. at an angle of 0°) relative to the load-bearing portion 204 by magnets. In accordance with other embodiments, electromagnets may be employed to selectively engage registration points as needed. For instance, electromagnets in one or both of the lightweight and loadbearing portions 202 and 204, or in/on respective surfaces of an interface 206 therebetween, may be activated to hold the lightweight portion 202 in place relative to the load-bearing portion 204 during static tests, and disengaged during tests in which user motion relative to a display is to be observed.

In accordance with yet other embodiments, the interface 206 between lightweight and load-bearing portions 202 and 204 may comprise a flexible material, such as a foam or elastic material. Such embodiments may further relate to a system in which there is a designated extent or range of motion that can be achieved between the different portions to accommodate user motion.

In some embodiments, the interface 206 between the lightweight and load-bearing portions 202 and 204 may comprise one or more force sensors. For example, and in accordance with one embodiment, an assessment may comprise asking the subject to focus on a moving target while maintaining their head motionless. If a user were to attempt to move their head during the test, and thereby attempt to move the lightweight subject-interfacing portion relative to the load-bearing portion, force sensors may then detect and/or quantify the subject's attempt to move their head up/down or side to side, thereby potentially indicating the presence of a cognitive impairment (or lack thereof).

In accordance with some embodiments, various sensors known in the art may be employed to monitor the relative movement of the lightweight and heavier supported portions of an assessment system. For example, such data may be acquired via proximity sensors or sensors monitoring an angle or change thereof between components of the respective portions. Further, such data may be complemented or supplemented by IMU motion data. For example, and in accordance with some embodiments, an IMU may be disposed on the lightweight portion of the assessment system in order to monitor unencumbered head motion. An IMU may alternatively, or additionally, be disposed on the heavier portion of the assessment system. For example, an IMU associated with the heavier portion may acquire motion data to capture noise for data filtering. In another embodiment, such motion data may be compared to motion data from an IMU on the lightweight portion for, for instance, smoothing or controlling for assessment variables. In accordance with another embodiment, an IMU disposed on the heavier portion supported by the subject's hands may monitor motion data to determine of a subject's hands are shaking, jittering, or exhibiting motion that may be indicative of a condition (e.g. a cognitive impairment). Alternatively, or additionally, such motion data may be employed to determine if a subject is generally moving (e.g. walking, on a moving platform, etc.), which may be enable safeguards for device usage. For example, a system may comprise a safeguard that will only enable use when it is determined from IMU data on one or more portions of the system that the subject is not moving.

Furthermore, and in accordance with various embodiments, an assessment system comprising a load-bearing portion and lightweight portion for minimising impact on a subject's ability to perform an assessment, or for improving assessment safety, may be configured such that heavier components are disposed within the load-bearing portion. That is, various embodiments relate to decoupling components that are required for performing light field-based cognitive assessment from an undue weight that may negatively impact assessments. For example, and in accordance with various embodiments, a portable HMD that is operable to perform a comprehensive set of tests may require multiple testing components and systems. For example, speakers, fluid for caloric tests, processing units for rendering light field content, and the like, may add an undesirable amount of weight to the system. Accordingly, the load bearing portion of an assessment system may be configured such that any heavier components, or components that are not required to be disposed on the lightweight portion for interfacing with the subject in order to perform an assessment, are disposed within the load-bearing portion to minimise the impact of weight on the subject and/or assessment, while maintaining the requisite components and functionality for an assessment system in a portable device.

In accordance with some embodiments, a lightweight portion 202 may comprise only those components necessary to assess user head motion (e.g. voluntary or involuntary) relative to the load-bearing or display portion 204. For example, and in accordance with some embodiments, the head-mounted portion 202 may comprise a means of fastening a position or motion sensor to the subject's head and the position or motion sensor itself. For example, an in accordance with one embodiment, an assessment system may comprise a motion sensor or a position sensor embedded in/on a strap or like means to be worn by the subject. It will be appreciated that such a sensor may include an inertial element (e.g. an IMU, accelerometer, or the like), and/or a positioning element, such as a radio frequency distance or positioning sensor. Accordingly, such a sensor may be operable to determine a relative position with respect to a corresponding component or frame of reference, or a change in position with respect thereto. For example, a position or distance sensor may be operable to detect a change in position with respect to a corresponding sensor on a load-bearing or display portion 204 of the system (e.g. one disposed similarly to the IMU 128 of the single-unit HMD 100 of FIG. 1G), thereby detecting and/or quantifying a user head motion during an oculomotor assessment.

As described above with respect to the HMD 100 of FIGS. 1A to 1E, the HMD 200 comprises a means of providing audio content to the user, schematically illustrated as headphones 208. The user-interfacing portion 202 may further comprise a pad for user comfort and/or isolating the subject's eyes from the external environment. Accordingly, while various embodiments relate to a system that removes any weight not required for performance of a cognitive assessment, various embodiments comprise additional elements on the lightweight portion 202, dependent on, for instance, the application and/or assessment at hand.

It will be appreciated that, in accordance with some embodiments, an assessment may comprise a step of calibrating the system with respect to user head position, for instance via placing the system (or the subject placing their head) in a designated position relative to one or more components of the system. For example, the assessment system may be configured to allow the subject to place their head in a designated position, so to enable the system to establish a baseline head position from which any deviations may be monitored. It will be appreciated that such a designated position may be established via one or more structural components of the system (e.g. a bar or form-fitting structure on the system), or by a digital calibration means wherein the subject and/or the system is maintaining stationary during a calibration procedure. Such calibration may be performed in accordance with the particular HMD configuration. For example, the single-unit HMD 100 of FIGS. 1A to 1J may comprise an IMU to calibrate overall device position and/or movement from a reference position. Such a calibration may similarly be performed with the HMD 200 of FIGS. 2A to 2D, while further employing a calibration process for calibrating the lightweight portion 202 relative to the load-bearing portion 204, in accordance with another embodiment.

It will be appreciated that, in accordance with some embodiments, such concepts may be similarly applied to light field-based assessment systems. For example, FIGS. 2E to 2H show yet another exemplary HMD 220 operable to perform an assessment. While various embodiments may comprise additional or alternative features, the assessment system 220 is equipped with wireless functionality and comprises a compact form factor for case of portability, while being robust for safe transportation and ready deployment in a variety of settings (e.g. school yard, football field, ambulance, race track, etc.). Accordingly, the system 220 is rugged, may be used outdoors, and may be easily and safely be transported. For instance, various embodiments relate to an assessment system that is light and compact, comprise a durable housing (e.g. ABS plastic), be fully enclosed, and comprise shock absorbent components and/or functionality, the nature of which will be appreciated by the skilled artisan. While HMD 220 may be schematically illustrated with bright (e.g. white) colours or textures for clarity, it will be appreciated that various embodiments relate to an internal enclosure system that provides a very dark environment and may accordingly be darkly coloured (e.g. black) in physical embodiments.

In some embodiments, the HMD 220 comprises light field generating functionality that may further comprise different form factors. For instance, and in accordance with at least one embodiment, a light field-based assessment device may be configured similarly to a phoropter, a non-limiting example of which is described in Applicant's co-pending U.S. Patent Application No. 63/013,304 entitled “Vision-Based Cognitive Impairment Testing Device, System and Method”, the entire contents of which are hereby incorporated by reference. However, for illustrative purposes, the HMD 220 comprises a small form factor which is readily transported for, for instance, rapid deployment in the field. In this case, the compact form factor of the light field-based HMD 220 is exhibited in FIGS. 2E to 2G, which show various views analogous to those presented above with respect to HMD 100 of external and internal components of the HMD 220. In this example, the HMD 220, as described above, comprises retractable earphone 222 to provide auditory stimuli.

The skilled artisan will appreciate that various other form factors similarly lie within the general scope and nature of the present disclosure. Similarly, the skilled artisan will appreciate that a light field-based assessment system 220 may comprise any components understood in the art as enabling the generation of a light field, examples of which may include, but are not limited to, one or more of a digital pixelated display screen, a (optionally dynamic, or dynamically actuated) light field shaping layer (e.g. a parallax barrier, a microlens array, a waveguide, a LCD screen, an aperture array, or the like), a digital data processor operable to, for instance, perform ray tracing calculations, govern pixel activations, adjust a light field shaping layer, or the like, and/or a plurality or combination thereof. Further discussion of exemplary light field generating elements and related processes herein contemplated may be found in, for example, U.S. Pat. Nos. 10,761,604. 10,394,322, and 10,636,116, and Applicant's co-pending U.S. Patent Application Nos. 63/056,188 and Ser. No. 16/992,583, the entire contents of which are hereby incorporated by reference.

In accordance with various embodiments, a light field-based assessment such as the HMD 220 may further comprise components related to the provision of various stimuli in addition to optical content. Indeed, it will be appreciated that various aspects related to, for instance, HMD 100 or HMD 200, as herein described (e.g. a two-part HMD comprising load-bearing and user-interfacing portions, one or more display screens operable to be displaced by one or more actuators towards or away from the user to, for instance, increase a range of depths at which content may be perceived by a user, speakers, caloric assessment components, or the like), may be similarly embodied in a light field-based testing system.

Returning now to FIG. 2E, for clarity of view of eye pieces 224, the exemplary embodiment of FIG. 2E shows a bare region 225 around eye pieces 224. However, various embodiments relate to the region 225 comprising a padding or flexible component (e.g. flexible rubber, not shown in FIG. 2E) for user comfort and stability during use, as described above. In accordance with some embodiments, such a padding may optionally additionally or alternatively serve as a means of blocking external light during performance of one or more assessments. A padding component may, in accordance with some embodiments, be disposable to, for instance, improve sanitation between tests and/or patients, or to be form fitting to different face sizes, or the like. It will be appreciated that such components (e.g. padding, eye pieces 224, or the like), in embodiments related to a two-piece HMD (e.g. HMD 200), but which employ light field technology to, for instance, present visual content in up to three dimensions for an assessment, may be disposed on a user-interfacing portion distinct from other aspects (e.g. display screen(s), light field shaping elements, or the like), which may be preferentially disposed on a load-bearing portion, in accordance with some embodiments.

In accordance with some embodiments of an HMD comprising light field technology, FIG. 2H is an image showing some of the internal components herein contemplated of the assessment device 220. In this example, device 220 comprises a light field generating system in turn comprising two distinct screens 226 for addressing each eye of the user. In accordance with one embodiment, screens 226 comprise two 5.5″ pixelated displays 226. Disposed between each screen 226 and eye piece 224 is a respective light field shaping layer (LFSL) 228, which, in this non-limiting example, comprises a microlens array (MLA) 228. In accordance with some embodiments, eye pieces 224 may further comprise respective lenses to further govern rays emanating from pixels of the displays 226 and LFSL 228 to produce the desired light field for a user. In some embodiments, lenses in eye pieces 224 may be tunable, for instance to increase a range of dioptric powers accessible to the system, or to accommodate a particular system geometry with respect to user eye position. In one embodiment, such lenses may comprise Optotune™ of like tunable lenses to, for instance, accommodate a wide range of perceptible depths that may be presented by the light field shaping system.

In accordance with some embodiments, one or more light field generating components may be tunable or adjustable. For instance, MLAs 228 may be translatable via one or more actuators to adjust the system geometry (e.g. moved towards or away from the display screens 226 or eye pieces 224) to enable, for instance, a wider range of dioptric power accessible to the system at an appropriate resolution for the application at hand and in view of the system properties (e.g. pixel density on screens 226, pupil-to-screen distance, or the like). Similarly, a head-mounted display system for performing an assessment may comprise other manually or automatically adjusted dynamic components to adjust system geometries. For example, electronic actuators or adjustable knobs may enable adjustment of various components to tune an eye relief distance, or tune for the interpupillary distance of a particular user. In some embodiments, the display screen(s) themselves may be dynamically adjusted towards or away from the user to enhance user experience and/or extend a range of dioptric corrections.

As described above, assessment device 220 further comprises, in this example, components enabling eye tracking functionality. For instance, device 220 comprises an infrared (IR) LED 230 for illuminating the eyes of a user for tracking during assessment using cameras 232. In accordance with various embodiments, the device 220 may comprise respective IR sources 230 and cameras 232 for each eye of the user. In other configurations, a single IR source 230 may illuminate both eyes while respective cameras 232 record individual eyes/pupils. The skilled artisan will appreciate that cameras 232 may be high-speed cameras, or be characterised by various specifications depending on the needs of a particular application, without departing from the general scope and nature of the disclosure. For example, and in accordance with one embodiment, cameras are mounted within the device and can track eye movements at rates greater than 120 Hz with an accuracy of less than 1° and a precision of less than 0.1°. In accordance with other embodiments, an accuracy of accuracy between 1 and 3 degrees and a precision of 0.1 to 2 degrees may enable assessments. The skilled artisan will appreciate that various embodiments herein contemplated relate to tuning of device parameters or components to adjust such accuracies and precisions based on the application at hand. For instance, accuracies and/or precisions may be increased or relaxed based on a desired frame rate and/or components of the device. In accordance with one embodiment, operation of an assessment system at a frame rate of 200 frames per second may enable a gaze precision of approximately 0.03 degrees, with an accuracy of 1 degree, based on the specifications of the gaze tracking components. In another embodiment employing different components and/or operating conditions, tracking may be performed at approximately 40 frames per second for an accuracy of approximately 1.3 degrees, and a precision of approximately 0.5 degrees. In accordance with one embodiment, a Pupil Labs camera and/or IR assembly may be employed.

The device 220, in accordance with various embodiments, may comprise various additional components depending on the application at hand. For instance, assessment device 220 of FIG. 2H also comprises an interpupillary distance sensor 234 for measuring the distance between a user's pupils to, for instance, improve a quality of a light field or light field perception for different users and/or user face sizes (e.g. calibrated for each new user of a system), and/or as a metric in various assessments (e.g. tracking the interpupillary distance during eye tracking exercises). Further, assessment device 230 further comprises an inertial measurement unit (IMU) 236 for measuring and reporting on user head movement during an assessment. Accordingly, an assessment device may comprise, additionally or alternatively, one or more, or a combination of, accelerometers, gyroscopes, magnetometers, or the like. Further, and in accordance with some embodiments, the assessment device 220 may comprise, without limitation, one or more additional cameras, accelerometers, temperature sensors, moisture sensors, or the like, operable to acquire, monitor, and/or report data. In some embodiments, such acquired data may be complementary to eye tracking data, as is common in, for instance, vestibular testing. For instance, a three-axis accelerometer may measure a user's head or body movement during tracking assessments to gauge a subject's coordinated eye and head movements, which may in turn be analysed to aid in the identification of potential neurological dysfunction. Conversely, the accelerometer may measure an abnormally high amount of head movement when a patient is asked or otherwise intended to remain stationary, which may be further useful in the diagnosis or assessment of various conditions. It will further be appreciated that while various aspects of an HMD have been described with respect to one or more of three exemplary configurations (e.g. HMDs 100, 200, or 220), any one or more aspects of one HMD presented may be applied to another of the HMDs described, in accordance with various embodiments. For example, an external status indicator for informing a practitioner of a user assessment may be similarly employed in light field-based HMD which comprises distinct load-bearing and user-interfacing portions, without departing from the general scope or nature of the disclosure.

Whether an HMD comprises a standalone unit (e.g. the HMD 100 of FIGS. 1A to 1J) or more than one component (e.g. the two-part HMD 200 of FIGS. 2A to 2D), or light field shaping functionality (e.g. HMD 220), various embodiments relate to an assessment device or method in which an HMD may be in communication either wirelessly or by a wired connection to external computational systems. For example, the HMD 100 of FIGS. 1A to 1J may comprise any necessary on-board computation resources or hardware components to enable wireless communication using known protocols (e.g. Bluetooth™, internet-based, or like communication protocols) with external devices, such as a medical practitioner laptop or desktop computer system or smartphone. Similarly, the HMD 100 may be coupled to such a resource via a wired or wireless connection, and may comprise any necessary components known in the art to enable same. In multi-component systems (e.g. HMD 200), such circuitry and/or electronic components may be disposed on, for instance, a load-bearing portion so to minimise undue weight on the user. In either case, it will be appreciated that such connectivity may enable, for instance, communication of assessment data, which may include, for example, metrics related to eye movement during the assessment, raw or filtered images or video of the user's eyes (e.g. live-streaming of the user's eyes) during assessment, as well as display of assessment stimuli or a representation thereof (e.g. what is displayed via a screen to the user during assessment). In accordance with yet further embodiments, such communication may enable remote control of the assessment from a practitioner. For example, a practitioner may control the presentation of a stimulus manually from a laptop or smartphone, which may be manifested in real time within the device to perform an assessment.

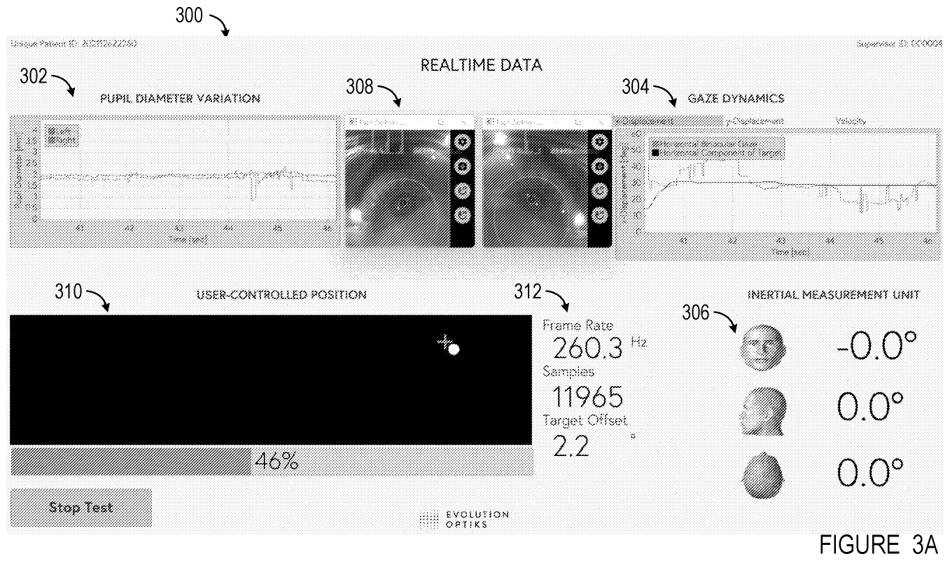

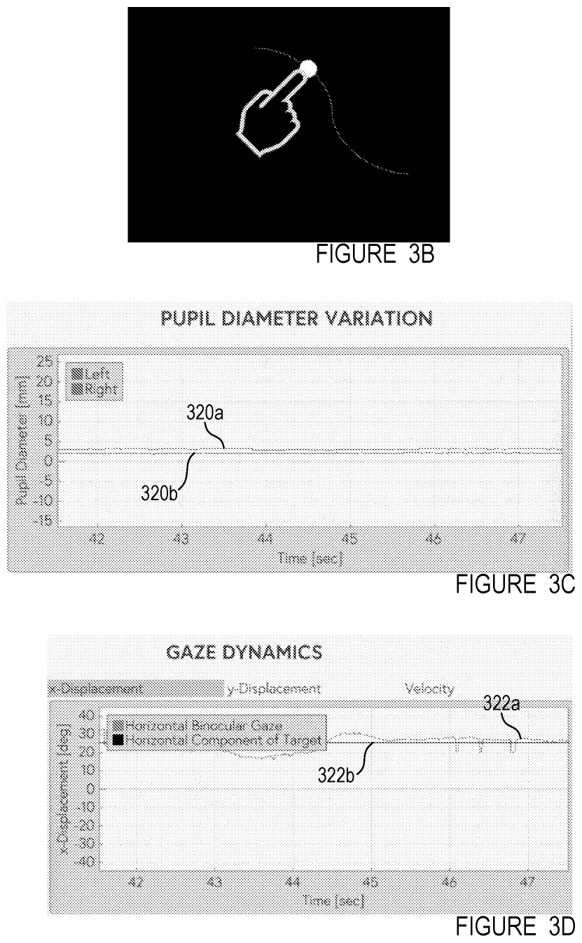

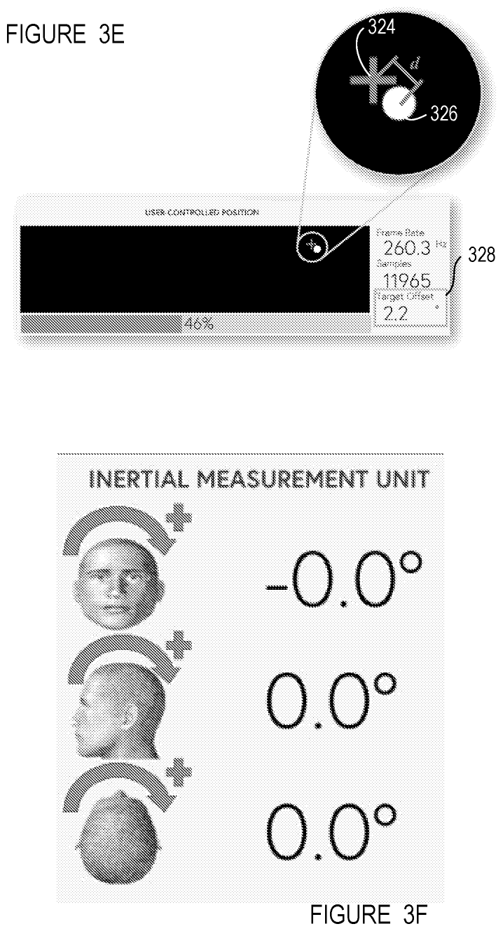

Such visualisation and control functionality may be provided via, for instance, a graphical user interface (GUI) associated with a practitioner device, such as a smart phone, tablet, laptop, or desktop computer. For example, FIG. 3A is a screenshot of an exemplary GUI for displaying various forms of information, as well as assessment control. In this case, the GUI displays real-time metrics related to pupil diameter variation 302, gaze dynamics 304 (e.g. gaze displacement, velocity, acceleration, or the like), as well as any head displacement or orientation 306. The GUI may further display video 308 of the user's eye(s) from within the HMD, as well as a representation 310 and/or manual control screen 310 of the display screen and/or stimulus as would be seen by the user of the device. Various monitoring parameters 312 may further be displayed and/or editable, such as a camera frame rate (e.g. reported as Hz, number of frames per second, or number of frames rendered per second), number of samples acquired, and/or any target offsets between where a target stimulus is presented relative to where the user is actually looking. Other features, functions or assessment data access may also or alternatively be considered, such as for example, digital access to a listing of assessed patients (i.e. database), and documentation, annotation and/or input recommendations associated therewith.

In accordance with various embodiments, an HMD may be configured to automatically execute assessments (e.g. through the automatic execution of digital instructions to perform the assessment, which may be stored on-board an HMD or accessed from a remote or connected system), or to provide manual control over an assessment by a practitioner. Such assessments may relate to, without limitation, saccade tests (e.g. predictive horizontal saccade tests, non-predictive horizontal saccade tests, predictive vertical saccade tests, and/or non-predictive vertical saccade tests), smooth pursuit tests (e.g. predictive or non-predictive horizontal or vertical smooth pursuit tests), reaction time tests, subjective visual tests, optokinetic nystagmus (OKN) tests (e.g. OKN horizontal and/or vertical tests), or vergence tests, to name a few.

Moreover, various embodiments relate to an HMD that may be pre-loaded or otherwise customised with a pre-set sequence of assessment. For example, a practitioner or manufacturer may store a battery or suite of designated assessments (e.g. specific vergence, saccade, and pursuit tests) in a designated order, on-board a device (e.g. a portable device for use in the field), depending on the application at hand. In some embodiments, this may be done before sale or shipping, or by a practitioner or like user. For example, a team doctor may customise a concussion screening protocol on a portable device for rapid deployment in the field, and may update the protocol as a season progresses, or as new tests become available, recommended, certified, and/or required by a governing body. In this case, the practitioner may connect to the HMD via a wired connection from their laptop, and interface therewith via a GUI, such as that described above. Alternatively, the practitioner or authority may remotely connect to a device to perform and desired updates to a testing protocol, for instance via the internet. Such wireless functionality may allow for ready updating of device software or firmware, allow for bidirectional communication of data or assessment parameters, and/or allow for customisability of assessments. For instance, and in accordance with some embodiments, various test suites may be uploaded, performed, and/or communicated on based on the application at hand.

For instance, in one embodiment, an assessment device may be primarily used in assessing a potential concussion in a racing environment (e.g. F1). The system may accordingly be remotely synchronised or preloaded (e.g. via a wired or wireless connection with cloud-based processing, or manufacturer- or otherwise installed) with a preferred or optimised profile of tests that are most appropriate or desirable to assess, for instance, cognitive performance for motorsports. Conversely, a system that is primarily to be used in a nursing home may present or be loaded with a different testing profile comprising, for instance, a different set of assessments to be performed. Similarly, a system for use in assessing a concussion risk during an NFL game following a head-to-head collision may be differently programmed, or contain different physiological sensors, than one used in a school or daycare facility. Accordingly, and in accordance with various embodiments, various aspects relate to a portable system that may, in some embodiments, comprise a comprehensive battery of tests (or instructions therefor) and/or sensing elements operable to perform generalised assessments for a wide range of cognitive functions, while other aspects may relate to customised testing routines for specific applications, with the ability to rapidly and easily update or exchange testing suites.

The performance of various examples of such assessments using an HMD will now be described, in accordance with various embodiments. While following aspects will be described with respect to the one-piece HMD 100 of FIGS. 1A to 1J for performing such exemplary assessments, it will be appreciated that such aspects may be similarly present in embodiments related to an HMD comprising load-bearing and lightweight portions, such as that of FIGS. 2A to 2D, and/or light-field shaping technology, such as the HMD 220, without departing from the scope or nature of the disclosure. It will be appreciated that, in accordance with various embodiments, the employ of a display screen without intervening optics in non-light field-based systems, such as those employed in VR, AR, or generally light field-based systems, and further the use of a wide-angle screen to present visual stimuli, such as the screen 106 of the HMD 100, may improve assessments by, for instance, increasing the angular range over which assessment may be performed.

However, it will be appreciated that various light field-based systems may be similarly employed to perform assessments, without departing from the general scope and nature of the disclosure. For example, Applicant's co-pending U.S. Application No. 63/179,021 entitled ‘Vision-Based Cognitive Impairment Testing Device, System and Method’, the entire contents of which are hereby incorporated by reference, describes various systems and methods using light field technology to assess a cognitive impairment.

In accordance with one aspect, there is provided an HMD for providing an assessment that is manually controlled by a practitioner. Such manual control provides the investigator with the freedom to control the position of a stimulus displayed within the device for focus and/or tracking by the user. This may provide the investigator with the ability to control the position, speed, acceleration, and direction of motion of, for instance, a stimulus that is displayed on a 2D display screen within the device (e.g. the stimulus 114 of FIG. 1I), or the particular LED that is activated in a vergence testing feature (e.g. the vergence testing feature 112 of FIG. 1J).

For example, one embodiment relates to the provision of user control over the presentation of a stimulus (e.g. white dot on an otherwise black or blank display screen) within the device, the position of which, in one embodiment, is controlled by via a user-controlled position control 310 of the exemplary GUI 300. In one example, the pointer is moved by the investigator within the allocated black-frame space within the GUI, wherein the corresponding stimulus may move accordingly within the device (e.g. on the display screen within the HMD). In accordance with some embodiments, there may be a designated and/or controllable correspondence between position on the control interface 310, and what is displayed on a screen within the device. For example, stimulus movement via the control 310 of the GUI 300 may result in a 1:1.8 correspondence of stimulus movement on the screen within the device. In an embodiment encompassing a single GUI for implementing and observing the results of a manually implemented test, the stimulus may be actively controlled via the screen interface (finger touch, mouse or pointer) while a recorded gaze variation is overlaid on the same plot. In automated test implementations, the target stimulus is moved along a predefined trajectory which can be displayed concurrently with the gaze tracking results. In accordance with another embodiment, the displayed stimulus may be controlled by a separate GUI from the GUI 300 shown in FIG. 3A, which may reserve the plot 310 for display purposes (e.g. to separately indicate user gaze with respect to a manually controlled target). In such embodiments, user control may be enabled via, for instance, a separate window on the practitioner device, or from a digital application on a smart phone, or the like.

In accordance with some embodiments, such manual control may be implemented to simulate various traditional assessments. For example, and without limitation, such a manually controlled assessment may be analogous to and/or conform with standards associated with traditional clinical methods of concussion diagnosis, whereby the investigator utilises a pen-like tool to stimulate the user's gaze to follow the position of the tool. While various tests may be automated, as will be further described below, such manual control may provide a practitioner greater control over stimulus position and/or movement than would be provided by an automatically repositioned stimulus. This may increase assessment flexibility, while simultaneously leveraging the experience of a practiced expert, wherein intuition or a particular cadence may help the practitioner in, for instance, establishing a diagnosis.

FIG. 3B shows one exemplary interface of a GUI for providing manual control over a stimulus displayed on a 2D screen within an HMD. In this example, the practitioner first designates that a manual test is to be performed, whereby and interface such as that shown in FIG. 3B allows the practitioner to place and move a white target displayed on a black background in a 2D setting, which may be seen by the user of the HMD on a screen therein, wherein the placement and motion of the stimulus is moved by, for example, a mouse pointer (e.g. in an embodiment where the HMD be connected in a wired fashion to a laptop), or a finger (e.g. in an embodiment where the HMD is in wireless communication with a tablet or smartphone).

During performance of a manual test, or indeed automatic assessments, some of which are further described below, various metrics may be monitored and/or reported, for instance via GUI 300. Similarly, various test parameters may be controlled or set via the GUI 300. Non-limiting examples of metrics monitored and user controls will now be described. It will be appreciated that any such metrics may be applied for different assessments below, as needed, and that some may enable the automatic performance of an assessment after being set by a practitioners, or being automatically set in accordance with a default value or range thereof, as appropriate.

In accordance with various embodiments, test controls may relate to the type and/or size of a stimulus presented via a display screen within the HMD. For example, a practitioner may designate the size of a circular target to be displayed, which may be manually entered or manipulated using a slider, button, or like mechanism on a GUI. Similarly, the practitioner may designate a test duration (e.g. 1 s to 600 s), or set any other number of control parameters, or ranges thereof.

Various real-time data or metrics may be reported via the GUI. For example, real-time pupil data may be reported as a pupil diameter, and may be identified as data streams corresponding to the left and right eyes. FIG. 3C shows an exemplary plot of pupil diameter variation during an exemplary assessment, wherein pupil diameters for the left pupil 320a and right pupil 320b are displayed for a designated time segment of an assessment.

FIG. 3D shows an exemplary GUI indicator displaying gaze dynamics. In this example, gaze position has been selected for display, whereby the angular position of gaze is displayed in units of degrees, and the practitioner has selected the horizontal (x) dimension of gaze position for display. The plot of FIG. 3D thus shows the horizontal component of binocular gaze 322a, as well as the x-component of the stimulus position 322b on which the user is to focus. In this example, the practitioner is also provided toggles to alternatively display the vertical displacement of binocular gaze and the presented stimulus, or gaze velocity, which may indicate an instantaneous velocity reported in, for instance, degrees per second.

FIG. 3D shows an exemplary GUI region displaying the user gaze position 324 and the stimulus position 236 as it appears on a screen in the HMD. Of interest for some applications is the target offset, reported as target offset 328 and schematically shown as d in FIG. 3E. In accordance with one embodiment, the target offset may be calculated as follows:

dₚₓ=\sqrt{(x_{G}−x_{T})²+(y_{G}−y_{T})²}

where dₚₓ is the target offset in pixels, which may be converted to degrees by computing:

$${d}_{deg} = {tan}^{- 1} ( \frac{Pixelsize * {d}_{px}}{D} ) * \frac{180}{π} * 2$$

where d_{deg} is the computed target offset in degrees, Pixelsize is the display-specific pixel size (e.g. 0.114 mm for the display screen 106 of the HMD 100), and D is the estimated distance in millimetres from the eye to the display (i.e. 141 mm for the configuration of the HMD 100).

The exemplary GUI 300 further reports head position in region 306 of FIG. 3A. Head position metrics may be indicated as, as shown in FIG. 3F, head roll, pitch, and yaw, reported in this case in units of degrees. It will be appreciated that other metrics may be reported, in accordance with other embodiments.

Having described several exemplary metrics, indicators, and controls associated with a GUI for controlling and/or observing oculomotor behaviour, various exemplary assessments will now be described. However, it will be appreciated that such assessments are presented for exemplary purposes, only, and that other assessments may similarly be performed, without departing from the general scope or nature of the disclosure. For example, various other oculomotor tests that may be similarly performed are described in Applicant's co-pending U.S. Patent Application No. 63/179,057.

In accordance with various embodiments, a head-mounted device (e.g. HMD 100, HMD 200) may be configured and operable to do perform saccade assessments, for instance for the purpose of screening for a potential TBI. In one example, a saccade assessment may comprise presenting a stimulus in the form of a white dot that appears in two different locations on a display screen within an HMD (e.g. the wide-angle display screen 106 of FIG. 1I). Such an assessment may be automatically performed, for instance via execution of digital instructions stored on the HMD or accesses by the HMD, in accordance with preset or designated parameters (e.g. assessment parameters defined in a GUI by a practitioner).

Saccade assessments may be performed in accordance with different axis modes, which may be selectable via a GUI, or pre-loaded as part of a predetermined battery of tests, wherein the positions of the stimulus may be aligned in either the horizontal or vertical axis, or along some other axis. In one exemplary assessment, a white dot is made to appear on a black background at a certain distance from the screen's centre for a designated amount of time before disappearing, to be relocated at the mirrored position along an axis such that the plane of reference passes through the display's center. Such a symmetric configuration relates to a predictive saccade test.

In accordance with one embodiment, the duration and location of the stimulus are based on a controlled computation of a square wave function derived from a sinusoidal wave function. For example, the desired position and duration of a stimulus presentation may be defined by the practitioner in the test controls region of a GUI, or predefined in accordance with a designated testing parameter set, to define the amplitude and period of the wave function, respectively. As a saccade test may require only one dot to appear at any given time at a fixed position during the entire duration of its appearance, the sinusoidal wave is replaced with a square wave function, in accordance with various embodiments.

In one embodiment, a saccade assessment may be predictive, wherein the amplitude of a square wave corresponding to stimulus position is constant, and the stimulus alternates between two fixed positions. And exemplary predictive saccade stimulus control function is shown in FIG. 4A, where the target position is shown as a function of time in the gaze dynamics region of the GUI. Generally, such a square wave function may be described with an angular frequency defined as w=2πƒ, and an amplitude A of:

$$A = \frac{D * tan ( \frac{{d}_{control} * π}{180} )}{Pixel Size}$$

where d_{control} is the pre-set displacement test control, Pixel size is the display-specific pixel size, and D is the estimated distance in mm from the eye to the display. To compute the square wave function governing the stimulus position over time, the stimulus displacement on a screen within the HMD may be given by the following, where t is the instantaneous time component calculated between two consecutive frames.

$$position of stimulus = \{ \begin{matrix}\frac{A}{2} & if 0 < sin ( w * t ) < 1 \\ \frac{- A}{2} & if - 1 < sin ( w * t ) < 0\end{matrix}$$

In accordance with some embodiments, non-predictive saccade tests may be similarly performed with an HMD comprising a display screen disposed therein. However, as non-predictive saccades relate to the appearance of stimuli in different positions, a random value may be introduced in the computation of the square wave amplitude A described above. For example, the amplitude calculation described above may be multiplied by a random number for each new stimulus position. In one embodiment, the random number is determined from a random number generator, wherein various device parameters are considered in the random number generation. For example, a random number may be generated within a specific range depending on, for instance, the size and resolution of a display screen. Alternatively, a random number between 0 and 1 may be generated, which is then multiplied by a maximum pre-set percentage of acceptable deviation from a designated position, which may then be scaled based on device parameter (e.g. width of a screen in pixels). FIG. 4B shows an exemplary plot of stimulus position during a non-predictive saccade assessment, wherein the stimulus displacement (y-axis) from a reference position (e.g. from display centre) changes in with each new position. It will be appreciated that either or both of predictive or non-predictive saccade tests may be similarly performed along any axis (e.g. horizontal, vertical, or other), and that various metrics related thereto may be monitored, recorded, and/or assessed. For example, a practitioner may be provided with metrics related to target offset changes in the user head position over the course of the assessment.

Another exemplary assessment that may be performed with an HMD as herein described relates to a smooth pursuit of a stimulus. As with saccade assessments described above, smooth pursuit assessments may be performed using an HMD comprising a display screen (e.g. the wide-angle display screen 106 of the HMD 100 described in FIGS. 1A to 1J).

A smooth pursuit assessment may similarly comprise the display of a stimulus (e.g. a white dot on an otherwise black or black display screen). In such an assessment, the stimulus is moved between two different locations along the display screen (e.g. between two points along a specific axis on the display). This may comprise, for instance, presented a white dot on a black background at the display's centre, whereby the dot then moves leftwards to a position specified by a displacement control (e.g. via a GUI). Upon reaching the defined destination, the point may then move rightward, and passing through the centre to reach a mirrored position.

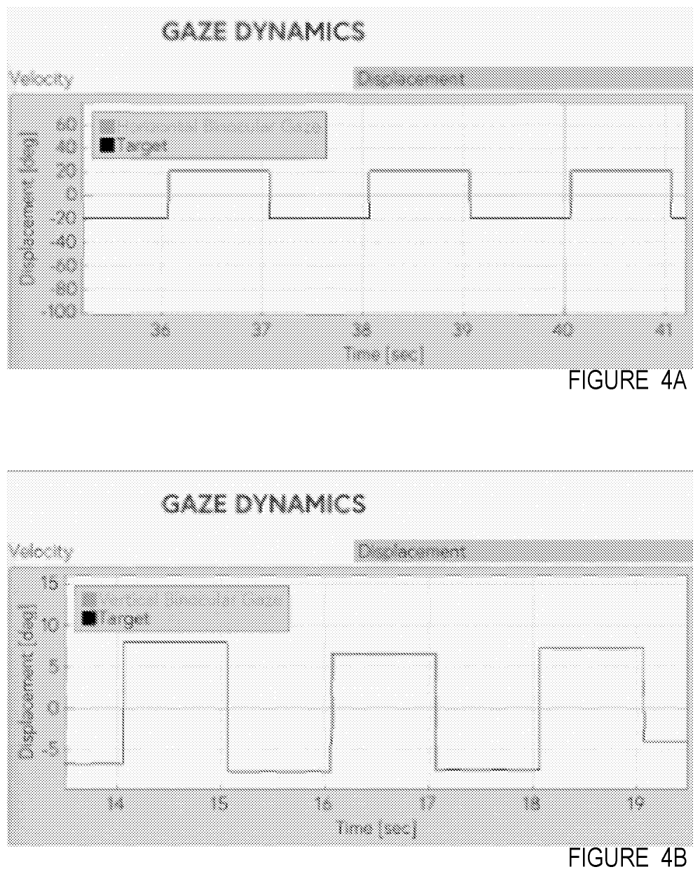

In one embodiment, this motion may be defined by a sinusoidal wave. Accordingly, the particular sequence of continuous positions of the stimulus may be defined by a controlled computation of the sinusoidal wave function. In other words, the position of the dot in during such an assessment is defined by the amplitude and period or frequency of the sinusoidal wave function defined by controls in a GUI or present in accordance with predefined testing parameters. As with saccade assessments, smooth pursuit may be predictive or not predictive (e.g. the amplitude of displacement changes between cycles), and may be performed along any designated axis. FIGS. 5A and 5B show exemplary sinusoidal functions defining stimulus position during predictive and non-predictive smooth pursuit assessments, respectively, performed using an HMD, in accordance with some embodiments. In this case, the amplitude of the sine wave of FIG. 5B changes upon each cycle based on the introduction of a random number in the calculation of the wave amplitude, as described above. FIG. 5C is an exemplary plot of a smooth pursuit assessment showing both the stimulus position, and the user gaze position as the user tracks the smoothly moving stimulus.

Various embodiments may further relate to and HMD operable to perform reaction time assessments. Such assessments may similarly relate the provision of a stimulus on a 2D display screen within the HMD, wherein, for example, a white dot appears on the black background for a short time (e.g. for 50 ms in the centre of the display screen). Such assessments may, in accordance with some embodiments, provide a potential biomarker for various conditions, such as a concussion, where concussed users often exhibit an increase in time required to react compared to baseline.

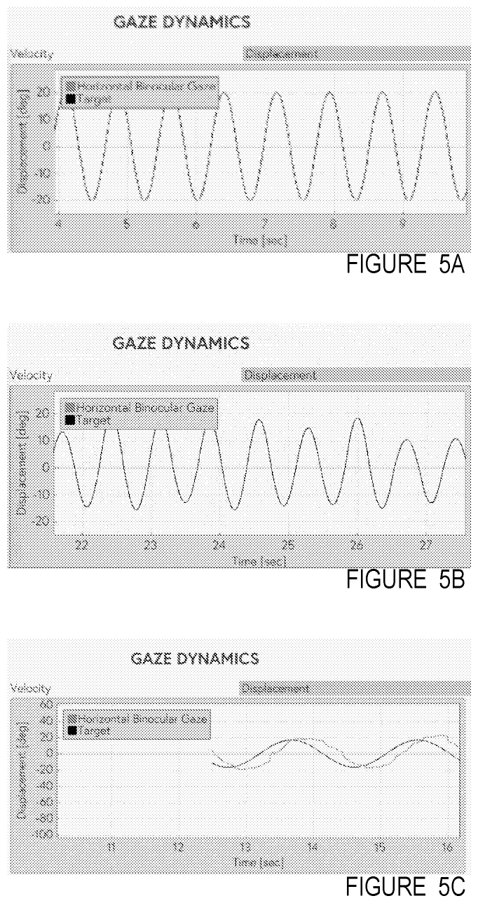

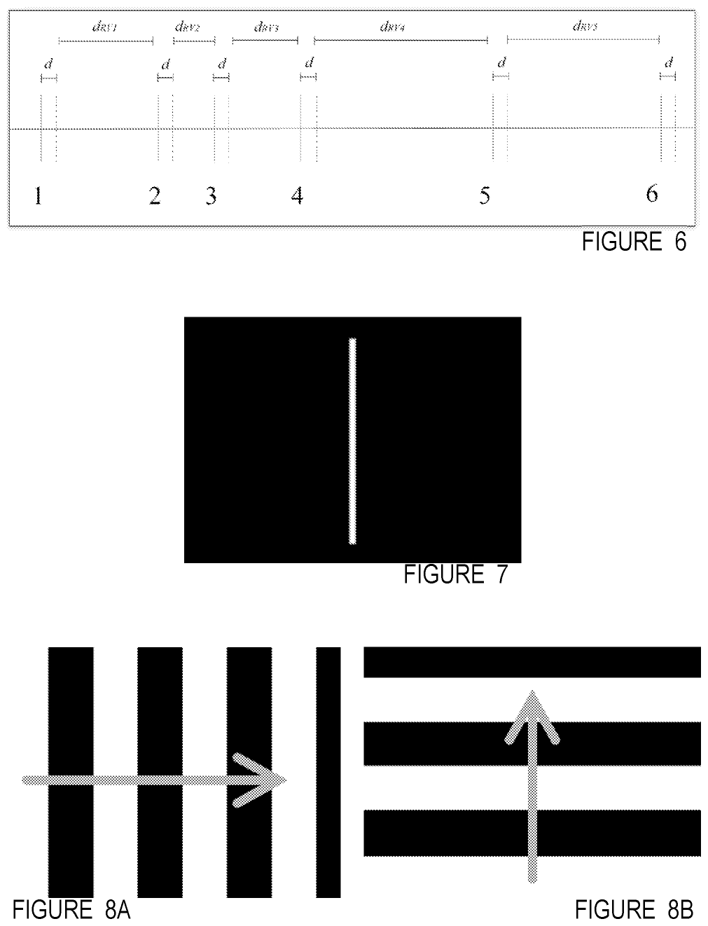

In one embodiment, the reaction time may be computed as the difference in time between a first frame rendering of the stimulus and the time at which a user performs a reaction, such as clicking a button (e.g. a mouse button, a keyboard button, or the like). Time may be recorded as, for instance, the difference in time stamps associated with these events. In accordance with some embodiments, one or more of the presentation time of the stimulus (i.e. how long a dot is presented for) and the delay time between successive presentations of the stimuli may preset, and may be fixed or variable. For example, a practitioner may define via a GUI a maximum duration time (or range thereof), as well as a maximum delay between successive events, while a randomly generated number is applied to these preset values to define random durations and delays. FIG. 6 is a plot illustrated one exemplary reaction time assessment, wherein a stimulus is presented six times for a duration d (e.g. 50 ms) at randomly defined intervals in time.

In accordance with various embodiments, an HMD as herein described may also enable the performance of subjective visual tests. For example, some embodiments relate to the digitisation of traditional light bar methods such as those used by Bohmer and Rickenmann, wherein a user is first disoriented (e.g. via the Dix-Hallpike manoeuvre) and asked to tilt their head until a bar a designated distance away and oriented at a designated angle appears to be vertically oriented or aligned with a particular axis. In a similar assessment, the initial position of the bar may also be oriented at a certain angle while the subject is asked to increase (or decrease) the angle (e.g. using keys of the keyboard) until they feel the line is perfectly horizontal or vertical.

In accordance with some embodiments, such assessments may be executed by rendering a stimulus (e.g. a bar) via the display screen at a designated orientation (e.g. 0.4 degrees), which may be adjusted by the practitioner. One example of such rendering is shown in FIG. 7. IMU components in the HMD may then provide data related to head orientation (e.g. yaw, pitch and roll), which may in turn be monitored via a GUI. some embodiments relate to assessments in which a user tilts their head until they feel that a line presented on the display screen is oriented, for instance, vertically or horizontally.

Various embodiments further relate to an HMD operable to perform optokinetic nystagmus (OKN) assessments. Such assessments of visual function may be particularly useful in, for instance, assessment of children or other users for whom obtaining a reliable response may be challenging. OKN assessments may relate to involuntary eye movement evoked by a repeating pattern stimulus in continuous motion. Such motion may consist of two phases: a smooth phase elicited when the user tracks a target (i.e. slow component velocity or SCV) and saccadic fast movement in the opposite direction (i.e. quick phase or QP), termed as a “resetting event”. This resetting event initiates when the user re-fixates on a newly appearing feature of the stimulus movement. The resulting data output is a sawtooth form when plotting displacement versus time. Various algorithms are known that are aimed at automatically extracting the resulting sawtooth data characteristics of gaze patterns, such as duration, amplitude and velocity estimates.

FIGS. 8A and 8B schematically represent OKN assessments rendered on a display screen of an HMD, in accordance with some embodiments. In this example, a pattern of alternating black and white rectangles with dimensions defined based on test controls set by a GUI or preset in accordance with default values are rendered. In some embodiments, such controls may relate to the spatial frequency of the pattern, and thus the number of rectangles shown in an angular range. Additionally, or alternatively, a practitioner may define a frequency to maintain while motion is sped up or slowed down. During assessment, the user is asked to fix their gaze on a black (or white) rectangle and follow its motion, indicated by arrows in FIGS. 8A and 8B. Once the gaze reaches the end of the display, the user moves their gaze back to the first colored bar of the pattern, and repeats following its motion.

Generally, the aforementioned assessments rely on the rendering of stimuli via a display screen within an HMD. As the position of stimuli is varied in up to two dimensions in the plane of the display screen over the course of an assessment, such stimuli may be considered to be dynamic stimuli. However, various embodiments herein described may additionally or alternatively relate to dynamic stimuli that move or appear to move in a third axis (i.e. towards or away from the user), without requiring the generation of a light field.

One example of such an assessment is a vergence assessment, provided via a vergence assessment tool disposed within the HMD (e.g. vergence assessment component 112 of HMD 100). A vergence test provides the practitioner with a tool that stimulates the user to move their eyes synchronously and symmetrically in opposite directions. If the motion of the eyes is towards the nose, it is known as convergence, and conversely, if the movement of the eyes is away from the nose and towards the ears, the movement is referred to as divergence. During such an assessment, in addition to the angular orientation of the eyes, the user must be able to adjust the eye's focus at the object located at the different distances, namely, accommodation. These two biological mechanisms work simultaneously to achieve a fast focused image at varying distances. If the movement is abnormal (e.g. asynchronous or with the same angular orientation/motion), the user would be identified as having convergence insufficiency (CI). Common biomarkers of CI include, but are not limited to, blurry vision, diplopia (i.e. double vision), near-sightedness, discomfort, nausea, discomfort, and eye fatigue (which are similarly most commonly observed following a trauma).

In accordance with various embodiments, a vergence test allows the user to look at equidistant axial points in space spanning from a far end of the display (e.g. the end of the display whereat a screen is disposed) to the nose. In some embodiments, this relates to a distance span of approximately 150 mm. This is achieved, in accordance with some embodiments, with an array of LEDs along a longitudinal axis of the HMD. FIG. 1J shows one such example, wherein a 1D array of LEDs 113 is disposed at along the upper inner side of the HMD 100, and is visible when worn by a user. In this case, the stimulus comprises light emitted by three 12-LED bars a linearly disposed end-to-end and integrated into the top inner surface of the HMD.

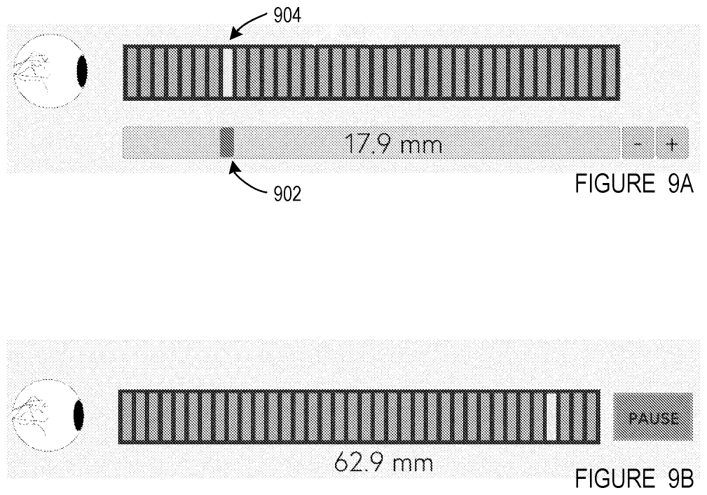

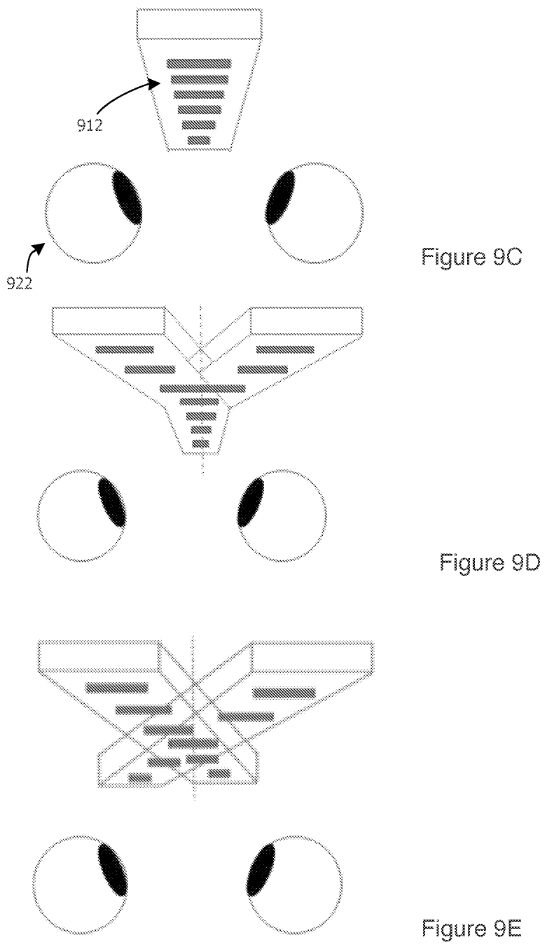

During assessment, LEDs of the array are sequentially lit and turned off, moving from the farthest point medially towards the nearest (or vice versa). The user is instructed to follow the light as it moves, and report on their experience (e.g. diplopia). In accordance with some embodiments, the sequential activation of stimuli (e.g. LEDs) may be manually performed by the practitioner, for instance via a GUI providing a control slider 902, the position of which corresponds to the LED to be current activated 904 (and therefore a distance between the active LED and the eyes of the user), as schematically shown in FIG. 1J. In such embodiments, the practitioner may, for instance, click and drag the slider, increase/decrease a distance using ‘+’ or ‘−’ icons, or the like. In accordance with other embodiments, the sequential activation of LEDs may be automatic (e.g. in accordance with preset assessment parameters), to, for instance, allow the practitioner to directly monitor user response (e.g. via video showing the user's eyes, or real-time plots of the user eye positions or velocities). In accordance with such an example, FIG. 9B schematically shows a GUI display corresponding to the position of the active LED in a sequence, while further offering a control button to manually begin or pause an assessment. In accordance with various embodiments, such assessments may further by executed in accordance with designated ‘speeds’ of activation, the direction of activation (i.e. towards or away from the eyes), a minimum or maximum distance to at which to provide illumination, or the like. Moreover, light sources may be activated in accordance with different sequences. For example, one assessment relates to the sequential activation of adjacent light sources, effectively incrementing the position of an activated light source one source at a time towards or away from the user's eyes (i.e. in a consecutive linear sequence). Other assessments comprise activating sources in accordance with different sequences that do no increment the active light source one position at a time. For example, one assessment may comprise an activation sequence in which a sources are activated at random (e.g. from a random position in the array of sources), or from alternating positions near and far from the user's eyes.

In accordance with various embodiments, FIGS. 9C to 9E schematically illustrate a vergence assessment using the 1D array of LEDs 902. While these schematics show similarly shaded rectangles corresponding to different LEDs, it will be appreciated that such LEDs are activated in accordance with a designated sequence (e.g. towards or away from the user). In this example, the user is asked to focus on each activated light source that appears to approach the eyes 922 of the user during the assessment.

In accordance with this embodiment, the vergence assessment comprises a vergence insufficiency test. For example, FIG. 9D schematically illustrates what a healthy user may perceive during a vergence insufficiency test. In this embodiment, the user's eyes 922 may sufficiently converge to properly perceive stimuli at least as far as a characteristic distance from their face. Conversely, attempts to focus on stimuli presented nearer than that characteristic distance may result in blurring, double-vision, discomfort, or other effects. A user with a cognitive impairment, however, may experience these effects even for stimuli that are presented at a distance further away than the characteristic distance, and/or may not be able to perceive or focus sources over a particular range of distances, as schematically represented in FIG. 9E. It will be appreciated that FIGS. 9C to 9E are presented for illustrative purposes (e.g. blurriness, double-vision), and do not necessarily represent what a user may experience.

It will be appreciated that various embodiments relate to the presentation of a vergence stimulus 902 in accordance with different testing protocols. For example, LEDs of the dynamic light source may be activated such that they are perceived as either approaching or retreating from the eyes 922 of the user. Furthermore, it will be appreciated that FIGS. 9C to 9E are shown for illustrative purposes, only, and that other configurations are herein contemplated. For example, similar systems and/or processes may be implemented in accordance with various medical requirements (e.g. those defined by the Food and Drug Administration) for a vergence test, without departing from the scope or nature of the disclosure.

It will be appreciated that the assessments described above comprise a non-exhaustive set of assessments that may be performed with an HMD as herein described. Moreover, HMD configurations such as those described above (e.g. comprising a display screen 106 and/or a vergence assessment component 112 or 902) are similarly presented for illustrative purposes, only, and that various other configurations are herein contemplated.

For example, a vergence assessment may be similarly performed using a light field shaping HMD, wherein visual content is provided via a light field shaping system within the HMD that is configurable to provide sufficient resolution and range of perceivable depths to adequately perform an assessment in accordance with standard or regulated practices. For example, a displaceable display screen may be employed within a portable light field-based HMD to perform a vergence test at high resolution, as further described in Applicant's co-pending International Patent Application No. PCT/US21/70944, entitled ‘LIGHT FIELD DISPLAY FOR RENDERING PERCEPTION-ADJUSTED CONTENT, AND DYNAMIC LIGHT FIELD SHAPING SYSTEM AND LAYER THEREFOR’, the entire contents of which are hereby incorporated by reference. It will be appreciated that such embodiments may further relate to the employ of, for instance, a GUI similar to that described above for controlling and/or monitoring various parameters, video, or data streams before, during, or after an assessment.

Further, various assessment devices relate to the provision of a dynamic visual stimulus (i.e. one that may by presented as being disposed or moving in 1D, 2D, or 3D within an HMD) via different means to elicit an oculomotor response of a user that is monitored via a gaze tracking system in the HMD for metric monitoring and reporting. Such stimuli may be provided via different means in up to three dimensions, in accordance with different embodiments of an HMD.

For example, in addition to a dynamic stimulus relating to the activation of different pixels on a static 2D display screen, and/or the provision of a 1D array of LEDs sequentially activated within the HMD, various other configurations and components are herein considered that relate to physical displacement of the stimulus itself, such as one presented via a displaceable screen, thereby providing 3D content or stimuli to further assess a patient response thereto presented at various depths. Such embodiments may overcome various drawbacks known to exist with light field, virtual reality (VR), or augmented reality (AR) systems, such as the vergence-accommodation conflict that may lead a user to experience fatigue, discomfort, or nausea.

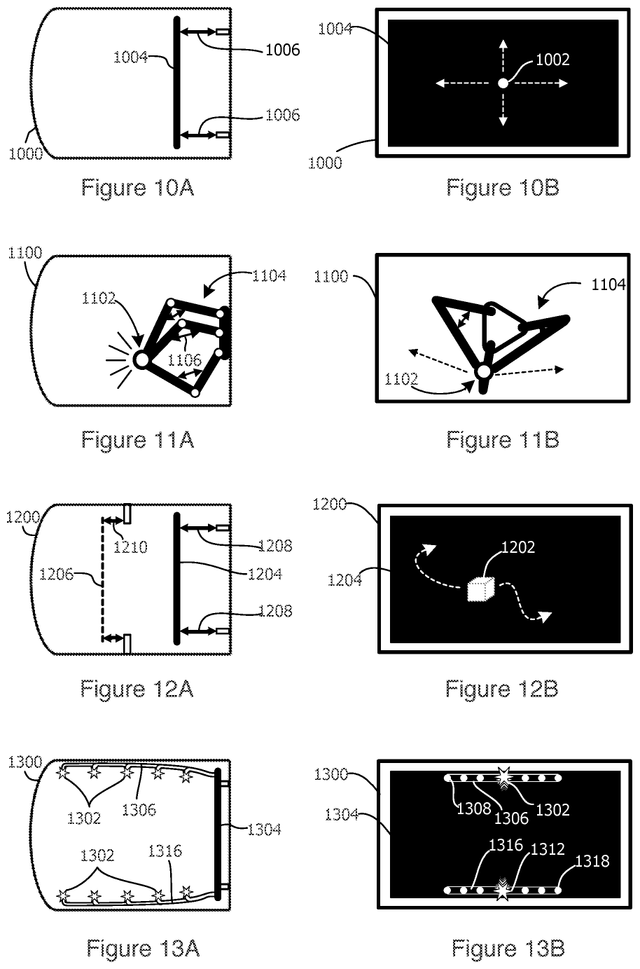

At least in part to this end, FIGS. 10A to 13B show various exemplary configurations for providing a dynamic visual stimulus to perform an assessment. For simplicity, the perspective of the load-bearing portion 204 of the HMD 200 of FIG. 2D is included in FIGS. 10A to 13B as respective assessment systems comprising different forms and configurations of dynamic stimuli. However, it will be appreciated that the load-bearing portion 204 is included in the embodiments described below for illustrative purposes, only, and that various other assessment configurations may be employed, in accordance with various embodiments. For instance, the following examples may be applied to the HMD 100, without departing from the general scope or nature of the disclosure.

For these exemplary embodiments, FIGS. 10A, 11A, 12A and 13A are schematics representing right side sectional views of different assessment systems comprising respective exemplary dynamic stimuli, while FIGS. 10B, 11B, 12B, and 13B are schematics representing front views (i.e. from the point of view of a user) corresponding to the side sectional views of FIGS. 10A, 11A, 12A and 13A, respectively. It will be appreciated that these schematics are not necessarily presented to scale, and that that only some components of exemplary assessment systems are shown for clarity and illustrative purposes.

As schematically illustrated in FIGS. 10A and 10B, one exemplary embodiment of an assessment system 1000 comprises a dynamic visual stimulus 1002 that is renderable via a display screen 1004 (e.g. a pixelated display screen 1004). In this non-limiting example, the display screen 1004 is coupled with the assessment system 1000, in this case a load-bearing portion 1004 of the system of FIGS. 2A to 2D, via actuators 1006 that are operable to translate or displace the screen 1004 closer to a user of the system (i.e. to the left in FIG. 10A, or out of the page in FIG. 10B), and/or further away from a user of the system (i.e. to the right in FIG. 10A, or into the page in FIG. 10B). Accordingly, such a system may be operable to perform, for instance, a vergence test. For example, a stimulus 1002 on a which a user focuses may be rendered on the screen 1004, and the screen 1004 may be translated towards the user while their eyes are monitored using an eye tracking system.

It will be appreciated that, in accordance with some embodiments, a dynamic visual stimulus may be re-rendered or otherwise adjusted during translation of the display screen 1004. For example, one embodiment relates to rendering a variably-sized stimulus (e.g. a visual stimulus that increases or decrease in size, a stimulus that changes shape or brightness, or the like) before, during, or after an assessment. Such a variably-sized stimulus may, for instance, improve a perception of an advancing or retreating stimulus during a vergence test, and/or improve a user comfort level while viewing to assist in mediating and sensory conflicts that a user may experience during assessments.

Furthermore, such a system may be operable to perform other vision-based assessments in addition to vergence tests. For example, the display screen 1004 may be operable to render a dynamic stimulus 1002 that appears to be moving in a plane characterised by the display screen 1004. For example, a saccadic assessment may be performed by sequentially rendering the dynamic stimulus at different regions of the display screen while the user's eyes are monitored. Similarly, a pursuit test may be performed by monitoring the user while the stimulus 1002 is rendered such that it appears to be moving across the screen 1004. It will be appreciated that such tests may be performed while the display screen is translated or displaced via the actuators 1006, thus providing a target stimulus 1002 that is moved, or has the appearance of moving, in three dimensions, in accordance with various embodiments.

In accordance with various embodiments, a dynamic visual stimulus provided by a display screen (e.g. screen 1004) as a rendered light source in an otherwise dark environment. For instance, and in accordance with one embodiment, all pixels of the display screen 1004 may be inactive (i.e. dark), while the stimulus is moved in up to three dimensions via one or more of a rendering sequence and activation of one or more actuators 1006 coupled to the display screen 1004.

In accordance with another embodiment, FIGS. 11A and 11B schematically show an assessment system 1100 comprising a stimulus 1102 that is dynamically adjustable in one, two, and/or three dimensions via a translation device 1104. In accordance with different embodiments, the translation device 1104 may comprise one or more actuators 1106, a robotic arm, a delta robot 1110, or the like, such that the dynamic stimulus may be translated, for instance, towards and/or away from a user. For example, the translation device may comprise an actuator operable to translate a stimulus along a track towards a user while the user's eyes are monitored, thereby performing a vergence test. Additionally, or alternatively, the translation device 1104 may comprise a plurality of actuators 1106 such that the stimulus 1102 may be translated in a plane (e.g. in two dimensions at a designated distance from the user's eyes) to perform a conventional pursuit assessment. The translation device 1104 (e.g. a delta robot 1104) may similarly be operable to rapidly reposition the stimulus 1102 such that the stimulus 1102 is perceived to have appeared at a new location, thereby enabling a saccade assessment. In accordance with some embodiments, the translation device 1104 may be operable to translate the stimulus 1102 smoothly and/or rapidly in three dimensions, thereby enabling various other assessments, or combinations thereof.

In accordance with different embodiments, the dynamic stimulus 1102 may comprise different elements or components. For example, a dynamic stimulus 1102 of a first embodiment may comprise an LED or other light source that is translatable in three dimensions via a delta robot 1104. In accordance with another embodiment, the dynamic stimulus may comprise a display screen that is translatable via a translation device 1104. For example, a small display screen (e.g. 1 cm×1 cm, 1″×1″, or the like) may be coupled with a robotic arm such that a stimulus 1102 may be rendered by the display screen to be tracked by the user while it is translated in up to three dimensions. Accordingly, through the employ of a small screen, a rendered stimulus 1102 may be physically translated in up to three dimensions, without requiring re-rendering or updating of pixel values to simulate movement. By comparison, the stimulus 1002 of FIGS. 10A and 10B may be rendered and/or moved on a display screen 1004 in two dimensions (i.e. the x-y plane) by way of refreshing displayed content, while the screen 1004 itself is translated in a third dimension toward or away from the user (i.e. the z-dimension or axis).

It will be appreciated that, in accordance with various embodiments, the assessment device 1100 may provide an isolated (i.e. dark) environment in which to perform a cognitive assessment. For example, the load-bearing portion of an assessment device may substantially eliminate ambient light during performance of an assessment. Accordingly, a dynamic stimulus may comprise a light source (e.g. an LED, activated pixels of a display screen, or the like) that is readily tracked by the user. Conversely, various other embodiments relate to the provision of a stimulus in a lit environment, such that a stimulus 1102 that is not inherently a light source may be seen and tracked by a user during an assessment. For instance, any object that is not a light source may be translated by a delta robot in ambient conditions in up to three dimensions while a user's gaze is monitored to perform a cognitive assessment.

Furthermore, it will be appreciated that, in accordance with various embodiments, a cognitive assessment system may reduce user discomfort by providing an assessment in conditions that are more natural to the user than are provided by, for instance, AR or VR systems. For example, a known challenge with VR systems is the occurrence of nausea or other symptoms as the user experiences conflicting sensory stimuli. It is herein contemplated that such conflicting stimuli may arise from as seemingly benign sources as ambient light, which may inevitably enter even an ‘isolated’ assessment system 1100, reflecting or otherwise interacting with system components, such as a display screen or pixels thereof, which are then perceived with negative effects by the user. Accordingly, various embodiments address such challenges through the use of alternative stimuli to those conventionally used in, for instance, saccade or pursuit assessments. For example, while conventional systems may employ relatively large static display screens to render content in two dimensions, some embodiments herein disclosed relate to the provision of a stimulus using a small display screen that has a majority of pixels activated to provide a visual stimulus. Accordingly, such embodiments may comprise a reduced amount of non-active pixels, glass, or the like, from which light may inadvertently be directed at the user and potentially cause discomfort. Similarly, a dynamic stimulus 1102 comprising a single, or a small number, of LEDs, or other light sources (e.g. fibre optics for guiding light) may be more benignly perceived by a user, even in the presence of stray light entering the system.

With reference now to FIGS. 12A and 12B, a further embodiment of an assessment system 1200 may comprise a light field display as a means of providing a dynamic stimulus 1202 for an assessment. In this example, the light field display comprises a pixelated display screen 1204 and a light field shaping layer (LFSL) 1206. In accordance with different embodiments, a LFSL may comprise, without limitation, a microlens array (MLA), a pinhole array, a parallax barrier, or other means known in the art, or a combination thereof, for shaping or governing a light field. Accordingly, it will be appreciated that various processes (e.g. ray tracing) and processing resources enabling the generation of a light field for performing a cognitive assessment may similarly be employed in accordance with various embodiments herein contemplated.

In the embodiment of FIGS. 12A and 12B, a dynamic stimulus 1202 for performing a cognitive assessment may be rendered via a combination of the display screen 1204 and LFSL 1206. While it will be appreciated that such systems may be operable to provide a 3D stimulus, and/or one that may be rendered to be perceived by a user as originating from one or more of a plurality of depth planes in, for instance, a vergence test, various embodiments may further relate to the provision of a dynamic stimulus via a translating or translated display screen 1204, and/or a translating or translated LFSL 1206. For example, one embodiment of an assessment system 1200 comprises a display screen 1204 that is coupled with the assessment system 1200 via one or more actuators 1208 operable to displace the screen relative to a user of the system and/or the LFSL 1206. Similarly, the LFSL 1206 may be displaceable via one or more actuators 1210 or like systems to translate the LFSL 1206 relative to the display screen 1204 and/or user. Accordingly, and in accordance with various embodiments, a cognitive assessment system 1200 operable to generate a light field may be operable to not only perform a vergence assessment, but may be operable to do so over, for instance, a wider range of rendered optotypes at a designated assessment resolution.

For example, a static light field shaping system may be operable to render a stimulus to be perceived at depth planes corresponding to a particular range of dioptric corrections that is limited by system components (e.g. MLA pitch and/or focal length, screen resolution, a spacing between the LFSL and screen, or the like). Conversely, a dynamically translatable display screen 1204 and/or LFSL 1206 may enable a wider and/or different range of dioptric corrections achievable for a system otherwise comprising the same components. Accordingly, and in accordance with various embodiments, a dynamic stimulus rendered via a dynamically adjusted display screen 1204 and/or LFSL 1206 may provide for a greater range of depth planes in a vergence assessment than would be accessible with a conventional static light field display for, for instance, a designated resolution of displayed content. In accordance with yet other embodiments, adjustment or dynamic displacement of a display screen 1204 and/or LFSL 1206 of a light field display may be employed to mitigate, for instance, vergence-accommodation conflicts that may hinder or otherwise give rise to user discomfort during a cognitive impairment assessment.

It will be appreciated that a dynamic stimulus in a light field-based assessment device 1200 may be rendered in different positions of the light field display to perform, for instance, saccade or pursuit tests, as described above. Further, various light field-based systems and methods herein disclosed may comprise integrated vision correction, and may enable correction of rendered content in accordance with a corrective eye prescription for a test or set of designated tests. Such corrections may be applied in addition to or alternatively to dioptric changes inherent in some tests (e.g. amplitude of accommodation tests). For example, and in accordance with various embodiments, a light field-based cognitive assessment may comprise the presentation of content to the subject in accordance with a perception adjustment designated so to accommodate a reduced visual acuity of the subject. That is, a conventional cognitive assessment targeting the oculomotor system may comprise presenting content (e.g. a test for assessing saccadic movement, smooth pursuit, etc.) at a fixed distance from the subject's eye(s) (e.g. from a 2D tablet screen or computer monitor), requiring a subject having a reduced visual acuity (e.g. farsighted, nearsighted, or the like) to wear prescriptive lenses to properly view the content. Conversely, various embodiments herein described relate to the operation of a light field assessment system 1200 for the presentation of content having a dioptric correction or optotype applied thereto (e.g. +3.0 D. −4.25 D, etc.). Accordingly, various embodiments allow the subject to properly view content without glasses or another form of corrective lenses, which would otherwise hinder the assessment by, for instance, interfering with eye tracking, inhibiting proper alignment of the device on the subject's face, or the like. Such content adjustments may be presented in addition to, for instance, dioptric corrections or image depth plane adjustments inherent in, for instance, a near point of accommodation or vergence assessment.

It will further be appreciated that while the application of such dioptric corrections may improve a quality or outcome of cognitive assessment tests, the dioptric correction required for a subject to clearly see displayed content may itself constitute a diagnostic test, in accordance with one embodiment. For example, a cognitive impairment assessment device 1200 may be operable to assess the visual acuity of a user through, for instance, the display of different optotypes. If a subject is observed to not exhibit a prior baseline of visual acuity, they may be exhibiting signs of a cognitive impairment.

In accordance with other embodiments, FIGS. 13A and 13B schematically show an assessment system 1300 comprising as a dynamic stimulus a series of light sources 1302 which, during a cognitive assessment, may be sequentially activated to simulate movement of a stimulus towards or away from a user of the system. In accordance with some embodiments, such a dynamic stimulus may comprise a series of LEDs or like light sources 1302 that may be activated such that sequential sources 1302 are activated as a function of decreasing or increasing distance from the user, as described above with respect the vergence assessment components 112 and 902. For example, a light source 1302 that is furthest from the user, or the rightmost source in FIG. 13A, may be first activated, then deactivated as the second rightmost source in FIG. 13A is activated, and so on, thereby simulating a dynamic stimulus that approaches the user. Such changes in position of the active source 1302, or simulated movement, may enable, for instance, a vergence test.

In contrast to the LED array 112 or 902 described above, in the embodiment of FIGS. 13A and 13B, light sources 1302 may be activated by way of a display screen 1304 with fibre optics 1306 or like waveguides disposed relative thereto so to direct light originating from pixels of the display screen 1304 to be emitted at various distances from the user. For example, a display screen may comprise designated pixels (e.g. a group or subset of pixels 1308) for providing light to a given fibre 1306 such that light will be emitted therefrom at a designated distance from the user upon activation of the designated pixels. A different pixel subset 1308 may emit light that is guided by a different fibre optic 1306, whereby it is emitted at a different distance from the user. In accordance with various embodiments, various assessments, such as a vergence assessment, may therefore be enabled via activation of different pixel groups 1308 over time such that different sources 1302 are presented to the user in accordance with the cognitive assessment.

In accordance with some embodiments, a display screen 1304 of a cognitive assessment system may have multiple sets of pixel groups corresponding to, for instance, different cognitive assessments. For example, the embodiment of FIGS. 13A and 13B comprise dedicated pixel sets 1308 in the upper region of the display screen 1304 that provide light to be guided by corresponding fibres and emitted in the upper region of the assessment device 1300. Such light sources 1302 may thus correspond to the provision of a vergence test generally in the upper field of view. Similarly, a dynamic stimulus 1312 may be provided in the lower field of view for a corresponding lower vergence test via fibres 1316 guiding light from corresponding pixel subsets 1318 in the lower region of the display screen 1304.

It will be appreciated that while five light sources 1302 are schematically shown in each of the upper and lower regions of the assessment system 1300 of FIGS. 13A and 13B, various embodiments relate to various numbers of light sources and configurations. For example, tens or hundreds of light sources 1302 may be provided in various configurations in 1D, 2D, or 3D space within the assessment device 1300, in accordance with different embodiments. In one embodiment, a three-dimensional distribution of LEDs may be disposed within the assessment system 1300 such that vergence tests may be performed as describe above, while saccade and pursuit tests may be similarly performed by sequentially activating different LEDs in a second or third dimension in the field of view. Similarly, additional fibre optics 1306 or 1316 may direct light from corresponding subsets of a pixels from a display screen 1304 in 1D, 2D, or 3D space within the assessment system 1300 to perform, for instance, various saccade, pursuit, and/or vergence tests.

In accordance with various embodiments, a display screen 1304 may comprise subsets of pixels 1308 designated to provide light to fibre optics 1306 or like elements to present a dynamic stimulus 1302, while other regions of the display screen may be employed for the provision of other visual content to the user. For example, one embodiment relates to the designation of upper and/or lower pixel rows of the display screen 1304 for providing depth-based dynamic stimuli to the user via corresponding optics 1306 for a vergence test, while the remaining display area may render stimuli for saccade or pursuit assessments. For instance, one embodiment relates to the designation of, for example, 4 to 10 rows of pixels near each of the upper and lower regions of the display 1304, while corresponding fibre optics 1306 are disposed to direct light from, within each of the designated 4 to 10 rows, respective subsets of 50 to 500 columns of pixels. The remaining pixels may then render a stimulus in accordance with, for instance, a saccade test, at a designated distance from the eyes of a user.

It will be appreciated that a display screen 1304 may further be coupled to, for instance, a casing of the assessment system 1300 via one or more actuators, such as the actuators 1006 of the assessment device 1000 of FIG. 10A. Accordingly, in addition to the provision of dynamic stimuli 1302 at various distances from the user, the assessment system 1300 may further provide content directly via the screen 1304 at various depths, in accordance with another embodiment.

Moreover, in accordance with various embodiments, an assessment system (e.g. assessment system 1000, 1100, 1200, or 1300) may comprise dark materials so to provide an isolated environment in which to perform an assessment. For example the inner casing of an assessment device may comprise a dark inner lining of a non-reflective material. thereby minimising stray light that may distract a user during an assessment, or provide a user with discomfort, as described above. For instance, the fibre optics 1306 of the assessment device 1300 of FIGS. 13A and 13B may be bundled within a dark casing extending from the screen to the user, while pixel subsets 1308 acting as sources for the dynamic stimulus 1302 may be similarly shielded and prevented from providing light that would otherwise by visible during an assessment. It will be appreciated that any other system components, such as processing resources (e.g. one or more digital data processors operable to execute digital instructions for performing ocular cognitive impairment assessment via a display screen, actuators, eye tracking systems, or the like), sensors, electronics, power sources, or the like, may be similarly encased and/or concealed within an assessment system.

It will further be appreciated that various embodiments relate to assessment devices comprising wireless functionality. For example, some embodiments relate to the provision of assessments as described above, while further providing assessment guidance via a display screen (e.g. screen 1004, 1204, or 1304) before, during, or after assessment. For example, an assessment system may comprise telepresence functionality to display a remote medical practitioner on a display screen during assessment as a picture-in-picture window. Accordingly, these and other embodiments herein contemplated may further comprise a microphone or like component to, for instance, communicate and/or record user responses or feedback during an assessment.

Returning again to the exemplary embodiments described with respect to FIGS. 1A to 1J (i.e. an HMD 100 comprising a wide-angle 2D display screen 106 and a 1D array of LEDs defining a vergence assessment component 112), the following description relates to various hardware specifications and configurations of an HMD for performing oculomotor assessments.

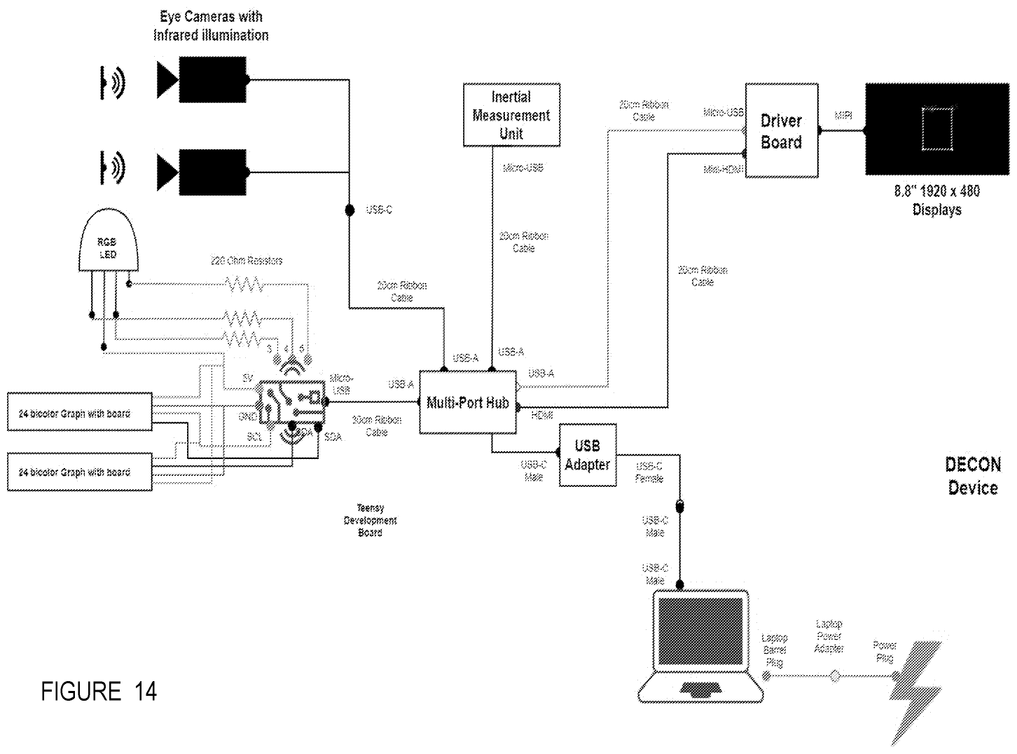

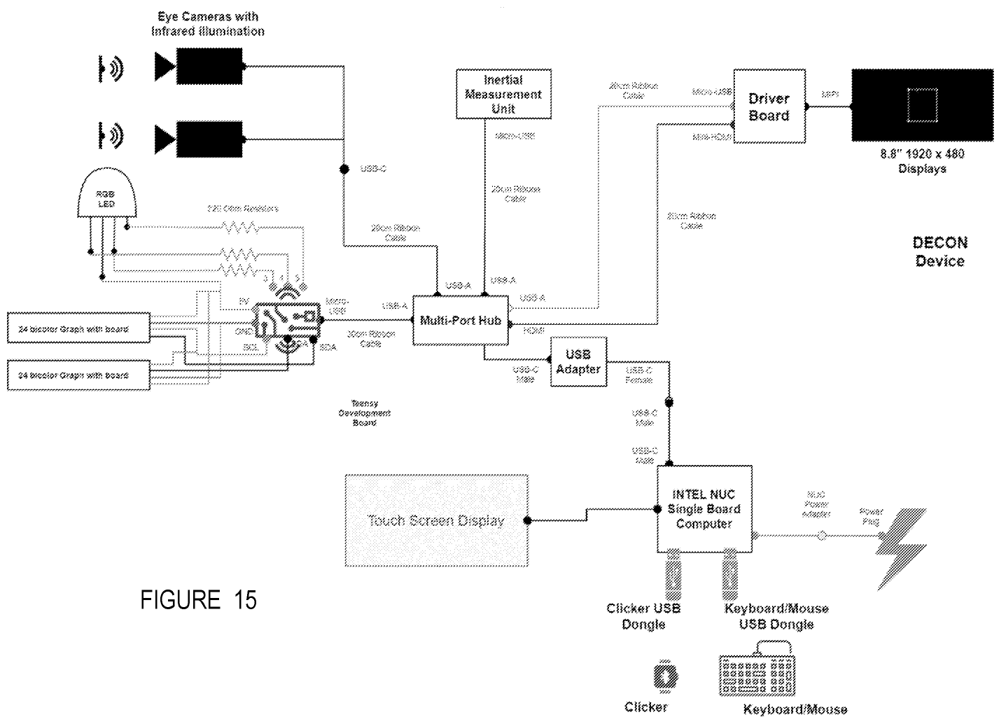

As described above, an HMD may communicate with a practitioner device through either a wired or wireless connection. In the case of a wired connection to, for instance, a practitioner laptop, the HMD may comprise the following non-exhaustive list of components, in accordance with one embodiment: an infrared-based embedded eye tracker for gaze extraction, an inertial measurement unit, a wide-angle display, a display driver board, a multi-port hub, a development board (e.g. a Teensy™ 3.2 development board), an RGB LED indicator (e.g. indicator 110), a vergence assessment component, also referred to herein as a ‘bar graph’, or ‘24-bicolour graph with board’ (e.g. vergence testing component 112), and a USB adapter. Such components may interact with a practitioner device operable to execute machine executable instructions (e.g. a digital application) on a practitioner device to perform various assessments. The interaction (e.g. connectivity) of such hardware components is schematically illustrated in FIG. 14, in accordance with one embodiment.

In embodiments of an HMD operable to perform assessments without a wired connection, the HMD may comprise the components described above with respect to the embodiment of FIG. 14, and may further comprise a single-board computer (e.g. an NUC computing device), a touch screen display, a remote control (e.g. a clicker with Bluetooth and/or USB dongle functionality), and/or a wireless keyboard and/or mouse, in accordance with one embodiment. An exemplary configuration and connectivity of various exemplary components of an HMD with wireless functionality is schematically shown in FIG. 15, in accordance with one embodiment.

With respect to hardware components, various aspects may be of consideration, depending on the application at hand. Accordingly, various specifications may be preferred for various embodiments of HMD configurations, and are hereby contemplated. For example, one non-limiting embodiment of an HMD comprises an embedded IR-based eye tracking system for gaze extraction having the following specifications: a tracking frequency of approximately 200 Hz, a field of view greater than approximately 100 degrees, a gaze accuracy of at least approximately 1.0 degrees, a gaze precision of at least approximately 0.08 degrees, a camera latency of approximately 8.5 ms or less, a processing latency of approximately 4 ms or less, an image resolution of approximately 192×192, at least a 5-point calibration method, and is operable in one or more of a stereo-mode, whereby both eye images are extracted to estimate binocular gaze, and a mono-mode, whereby each eye image is extracted to estimate monocular gaze. Such specifications are non-limiting. For example, in accordance with one embodiment, an embedded IR gaze tracking system may comprise cameras operating at 30 Hz with a 400×400 image resolution. In accordance with one embodiment, an embedded IR gaze tracking system comprises a Pupil Labs™ HTC Vive Add-on set.

In accordance with one embodiment, an embedded IMU within an HMD may relate to the following non-limiting specifications, in accordance with one embodiment: a resolution of less than approximately 0.01 degrees, an orientation range corresponding to roll (±180°), pitch (±90°), and yaw (±180°), static and dynamic accuracies of less than approximately 0.5 degrees and 2 degrees, respectively, a data transition rate of up to approximately 400 Hz, a 3-axis accelerometer, a 3-axis magnetometer, and a 3-axis gyroscope. In one embodiment, an embedded IMU is set to operate at a transmission rate of 100 Hz and extracts the Euler angles during the various assessments. In one embodiment, the embedded IMU comprises a Xikaku™ LPMS-B2 model.

In accordance with one embodiment, wide-angle display within an HMD may relate to the following non-limiting specifications, in accordance with one embodiment: a diagonal size of approximately 8.8″, a 1920×480 pixel format, a 218.88×54.72 mm (H×V) display area, an aspect ratio of at least 3:1, a pixel pitch of approximately 0.114×0.114 mm (H×V), a brightness of approximately 600 cd/m², a contrast ratio of approximately 800:1, approximately 16.7M (8 bit) colour numbers, a USB power of 5 V, and a refresh rate of approximately 60 Hz.

In accordance with various embodiments, a multiport hub may comprise various configurations. In one non-limiting embodiment, a multiport hub comprises four USB-A hubs, an HDMI hub, and a USB-C hub.

In order to facilitate the assembly process of an HMD and reduce cabling within the device, as well as to reduce the risk of disconnection, various embodiments relate to the employ of custom-designed PCBs installed within an HMD. For example, the connections between the RGB LED indicator, bar graph (i.e. vergence assessment component), and Teensy 3.2 development board in FIGS. 14 and 15 may be electrically coupled via custom-designed PCB boards. For example, a PCB circuit board was designed for electrically coupling three 12-LED linear arrays (i.e. to form a vergence assessment component 112) such that four single outputs (SCL, SDA, VCC, and GND) are couplable to a Teensy development board. The Teensy development board is in turn comprises an on-board microprocessor (e.g. an Arduino™ or like microprocessor) for executing digital instructions compatible therewith. In accordance with some embodiments, this latter board may serve to, among other aspects, power the former PCB board.

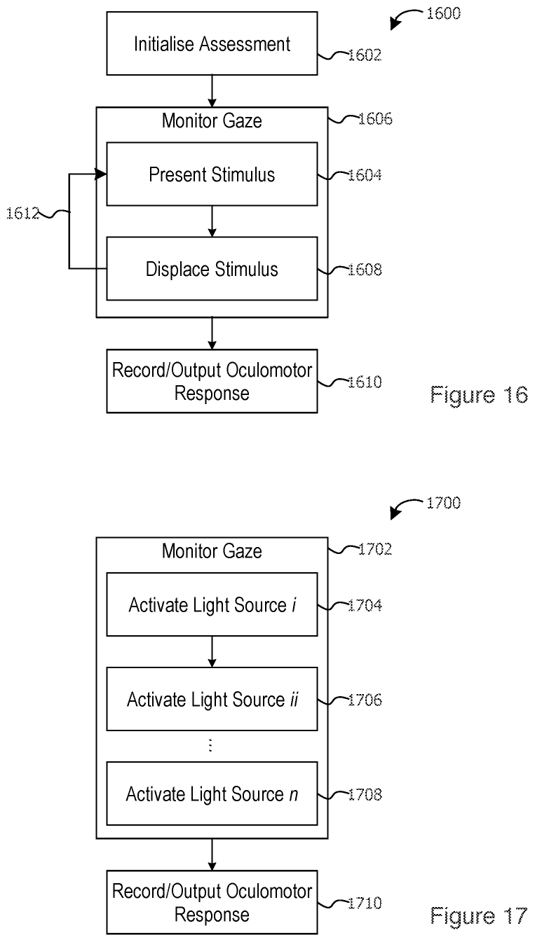

With reference now to FIG. 16, and in accordance with one exemplary embodiment, a method for performing an assessment of a user, generally referred to with the numeral 1600, will now be described. In this example, the process 1600 may be executed using a digital data processor in communication with an eye tracking system configured to monitor an oculomotor response to a stimulus that is presented to the user in accordance with an assessment. The processor may further be in communication with one or more digital data storage devices having stored thereon digitally executable instructions for performing one or more assessments, non-limiting examples of which may include vergence, saccade, and/or pursuit tests. The processor may further be in communication with one or more components of the stimulus, such as a light source or plurality thereof (e.g. a pixelated display screen, an array of LEDs, or the like), one or more actuators, operable to displace the stimulus, or the like.

The process 1600 may comprise an initialisation step 1602 in which the assessment system is configured to begin a designated assessment. For example, a delta robot (e.g. the HMD 1100 of FIG. 11) may translate the stimulus to an initial position far away from the user to begin a vergence test. The process may then comprise the presentation 1604 of the stimulus to the user, such as the activation of an LED or pixels of the display screen via the digital data processor, during which time an eye tracking process and/or system may monitor 1606 the user's gaze, pupil, or eye positions (or movement thereof).

In accordance with various embodiments, an assessment device may then displace 1608 the stimulus in accordance with the designated assessment while a user response is monitored 1606 via a gaze tracking system or process. For example, the translation device may displace 1608 the stimulus in a first dimension towards the eyes of the user (i.e. in the z dimension or direction). As the user focuses on the approaching stimulus, the degree of vergence of the user's eyes may be monitored 1606 by the system, wherein the vergence response may be recorded 1608 and/or output 1608 for diagnostic purposes. For example, a signal corresponding to raw or minimally processed gaze data may be recorded and output 1608 for further analysis by a medical professional. In accordance with another embodiment, recorded data may be analysed by the system to assess, for instance, a risk of a cognitive impairment, for instance by comparing the ocular response of the user to a baseline or other dataset (e.g. aggregate data of healthy and impaired individuals). A signal representative of the user's oculomotor response, such as a digital signal corresponding to gaze accuracy, smoothness, lag to a moving stimulus, or the like, either in raw or processed form, may be output 1608 (e.g. displayed on an interface, transmitted to a remote device for review by a medical practitioner, or the like), for further assessment, in accordance with various embodiments.

In accordance with some embodiments, stimulation presentation 1604 and displacement 1608 may comprise sequential rendering of stimuli in various positions on a wide-angle display (e.g. display screen 106) in accordance with a designated 2D assessment, or sequentially activating LEDs of a vergence assessment component (e.g. vergence assessment component 112).

In accordance with some embodiments, the presentation 1604 and displacement 1608 of a stimulus may be repeated 1612 as desired to, for instance, acquire sufficient statistics to adequately assess the user for a cognitive impairment. Alternatively, or additionally, the process steps of presenting 1604 and displacing 1608 the stimulus schematically shown in FIG. 16 may be iterated in the performance of alternative assessments. For example, the process 1600 may be employed to perform a saccade or pursuit test, in accordance with some embodiments. For instance, a saccade test may comprise the steps of initialising 1602 the assessment by placing the stimulus to the right side of the assessment system. The system may then present the stimulus 1604 to the user in this first position, where it is maintained for a designated amount of time. The stimulus may then be rapidly displaced 1608 (or rerendered 1608) by the translation device (or by a wide-angle display screen 106) in a second position (e.g. to the left of the user in the x and/or y dimension or direction) while the user's gaze is monitored 1606 to assess an oculomotor response. The process may then repeat 1612 as the stimulus is dynamically moved in the second dimension (e.g. back and forth from left to right in the x direction) in accordance with the saccadic assessment. It will be appreciated that, in accordance with some embodiments, such repetition 1612 may comprise the deactivation of the stimulus (e.g. extinguishing a light source or display screen) while the stimulus is displaced, such that it may be presented 1604 in each new position. However, in accordance with other embodiments, the translation device may displace 1608 the stimulus such that its movement is imperceptible to the user (e.g. faster than what is observable by the user, or instantaneously rerendered in a new position).

Similarly, a vertical saccade test may be performed by displacing the stimulus 1608 in a third dimension (e.g. from up to down in the y dimension). It will be appreciated that various embodiments relate to the displacement 1608 of the stimulus in more than one dimension (e.g. in 3D) during an assessment via the translation device.

It will be appreciated that pursuit assessments may similar be performed in accordance with the process 1600 by, for instance, more slowly displacing 1608 a presented stimulus 1604 in up to three dimensions while user gaze is monitored 1606, in accordance with various embodiments.

While such description may relate to physical displacement of a stimulus, it will be appreciated various embodiments relate to similar processes employing light field-based systems configured to present stimuli in up to three dimensions. For example, a light field display may execute the method 1600 for 2D assessments (e.g. a 2D assessments where in visual content is rendered to be perceived at a designated image plane), and/or may present a stimulus at different depth planes during an assessment to perform, for instance, a vergence test.

In accordance with other embodiments, and with reference to FIG. 17, another exemplary process for performing an ocular cognitive impairment assessment of a user, generally referred to with the numeral 1700, will now be described. In this example, the process 1700 relates to the performance various cognitive assessments using a plurality of light sources that are independently addressable by a digital data processor such that a visual stimulus may be provided to the user at a plurality of physical positions relative to the user, wherein each physical position is associated with a corresponding one of the plurality of light sources (e.g. using the vergence assessment component 112 comprising a 1D of LEDs that are independently addressable). While description of the process 1700 will be provided with respect to the assessment device 600 of FIGS. 6A and 6B comprising a display screen, the pixels, or subsets of pixels, of which may constitute the plurality of light sources, the light from which may be directed to different physical locations via, for instance, fibre optics, it will be appreciated that various embodiments may relate to the use of, for instance, LEDs disposed within an assessment system at respective physical positions in up to three dimensions such that they may sequentially activated in accordance with a designated cognitive assessment.

In accordance with some embodiments, the vergence assessment comprises light sources at different physical positions, which, when activated in accordance with a designated sequence, may act as a dynamic light source (e.g. a light source that appears to be moving or have changed positions) during an assessment. Accordingly, a method 1700 for performing an ocular cognitive assessment may comprise the execution of digital instructions by a digital data processor to activate LEDs of a vergence testing component in a sequence such a light source will be presented dynamically in up to three dimensions over the duration of an assessment.

For example, the process 1700 may comprise monitoring the user's gaze 1702 while a first light source is activated 1702 (e.g. a first LED is activated 1702), wherein the user focuses on the physical position where light is observed. For a vergence assessment, this may relate to the activation of the LED that is at the farthest position from the user.

The digital data processor may then activate a second light source 1706, the light from which is observable by the user as being in a different physical location than that observed from the first light source. Continuing with the example of a vergence assessment, this may correspond to the activation of a second LED disposed at the second furthest location from the user (i.e. the second furthest light source in the z-axis of the system). It will be appreciated that the digital data processor may continue to maintain activation of the first light source 1704 during activation of the second light source 1706, in accordance with one embodiment. However, various other embodiments relate to the extinguishing of the first light source prior to, simultaneously with, or after activation of the second light source 1706.

The process 1700 may continue with activation of different light sources 1708 corresponding to different physical positions relative to the user. Upon completion of an assessment, any raw or processed gaze tracking data may then be recorded and/or output 1710 as a signal corresponding to a user response to the dynamic stimulus provided by the sequential activation of light sources, as described above.

It will be appreciated that various assessment may be performed in accordance with the process 1700. For example, a vergence test may be performed by sequentially activating light sources that approach or retreat from the user in a first axis. Conversely, a saccade assessment may comprise a different sequence of light source activation, wherein light sources are activated sequentially on different sides of the field of view in a second direction (e.g. left and right), and/or a third dimension (e.g. up and down). Similarly, light sources observable in adjacent physical locations (e.g. subsets of pixels light is emitted by fibre optics with adjacent emitting ends) may be activated in sequence so to provide the perception of a moving source during a pursuit experiment. It will further be appreciated that such assessments may be performed in up to three dimensions, even within the same assessment.

It will further be appreciated that the process 1700 may be employed for a combination of assessments. For example, a vergence test may be performed as described above by activating subsets of pixels such that a dynamic visual stimulus approaches a user. A saccade test may subsequently be performed using other pixels of the display screen that are directly visible to the user, wherein a first light source is activated 1704 (e.g. a subset of directly visible pixels is activated 1704) on the right-hand-side of an assessment system, followed by the activation of a second light source 1706 (e.g. a second subset of directly visible pixels is activated 1706) on the left-hand-side of the system.

Furthermore, it will be appreciated that various combinations of elements herein described are also herein contemplated. For instance, an assessment system executing process 1700 of FIG. 17 may further employ one or more actuators operable to displace a light source (e.g. a pixelated display screen) in a first direction (e.g. towards or away from a user) so to, for instance, control a plane at which saccade or pursuit tests are performed, while pixel subsets coupled with corresponding fibre optics may enable a greater range of stimulus depths for a vergence test.

While the present disclosure describes various embodiments for illustrative purposes, such description is not intended to be limited to such embodiments. On the contrary, the applicant's teachings described and illustrated herein encompass various alternatives, modifications, and equivalents, without departing from the embodiments, the general scope of which is defined in the appended claims. Except to the extent necessary or inherent in the processes themselves, no particular order to steps or stages of methods or processes described in this disclosure is intended or implied. In many cases the order of process steps may be varied without changing the purpose, effect, or import of the methods described.

Information as herein shown and described in detail is fully capable of attaining the above-described object of the present disclosure, the presently preferred embodiment of the present disclosure, and is, thus, representative of the subject matter which is broadly contemplated by the present disclosure. The scope of the present disclosure fully encompasses other embodiments which may become apparent to those skilled in the art, and is to be limited, accordingly, by nothing other than the appended claims, wherein any reference to an element being made in the singular is not intended to mean ‘one and only one’ unless explicitly so stated, but rather ‘one or more.’ All structural and functional equivalents to the elements of the above-described preferred embodiment and additional embodiments as regarded by those of ordinary skill in the art are hereby expressly incorporated by reference and are intended to be encompassed by the present claims. Moreover, no requirement exists for a system or method to address each and every problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, as may be apparent to those of ordinary skill in the art, are also encompassed by the disclosure.

1.-32. (canceled)

33. A head-mountable device for performing an oculomotor assessment of a user, comprising:
a widescreen display to be disposed, when the device is mounted, in direct unrefracted line of sight to render a dynamic visual stimulus horizontally displaceable in a wide binocular field of view to stimulate a wide field of view oculomotor response thereto, wherein said widescreen display is physically mounted within a viewing tunnel that optically isolates, when mounted up against the user's face, viewing of said widescreen display;
an eye tracking system configured to monitor said wide field of view oculomotor response; and
a digital data processor in communication with said widescreen display and said eye tracking system and operable to execute digital instructions for performing the oculomotor assessment by:
activating said widescreen display to horizontally displace said dynamic visual stimulus in accordance with the oculomotor assessment; and
recording said wide field of view oculomotor response.

34. The device of claim 33 wherein said digital data processor is further operable to execute digital instructions for outputting an assessment result indicator as a result of said wide field of view oculomotor response.

35. (canceled)

36. The device of claim 33, wherein said viewing tunnel comprises a substantially amorphous internal surface to reduce internal reflections.

37. The device of claim 36, wherein said amorphous internal surface is at least partially provided by a fabric.

38. The device of claim 332, wherein said wide binocular field of view comprises a horizontal field of view of at least 65 degrees.

39. The device of claim 38, wherein said horizontal field of view is of at least 70 degrees.

40. The device of claim 33, further comprising a plurality of light sources disposed to project inwardly along an axis perpendicular to a plane of said widescreen display and operable to present a visual stimulus at a corresponding plurality of physical locations at respective relative distances to the user along said axis.

41. The device of claim 40, wherein said plurality of light sources is disposed along an upper viewing tunnel surface extending from above said widescreen display toward the user.

42. The device of claim 40, wherein said plurality of light sources is activated to test a near point of convergence.

43. The device of claim 33, wherein said eye tracking system comprises at least one tracking light source oriented to illuminate the user's eyes, and at least one camera oriented to capture a response of the user's eyes to illumination from said at least one tracking light source.

44. The device of claim 43, wherein said at least one tracking light source comprises an infrared (IR) light source, and wherein said at least one camera is at least sensitive to IR light.

45. A system for performing an oculomotor assessment of a user, the system comprising:
a head-mountable device comprising a widescreen display to be disposed, when the device is mounted, in direct unrefracted line of sight to render a dynamic visual stimulus horizontally displaceable in a wide binocular field of view to stimulate a wide field of view oculomotor response thereto, and an eye tracking system configured to monitor said wide field of view oculomotor response, wherein said widescreen display is physically mounted within a viewing tunnel that optically isolates, when mounted up against the user's face, viewing of said widescreen display;
a digital data processor in communication with said widescreen display and said eye tracking system and operable to execute digital instructions for performing the oculomotor assessment by:
activating said widescreen display to horizontally displace said dynamic visual stimulus in accordance with the oculomotor assessment;
recording said wide field of view oculomotor response; and
outputting an assessment result indicator as a result of said wide field of view oculomotor response.

46. The system of claim 45, further comprising an operator application digitally executable on a distinct operator device having a digital display screen and a communication interface to said head-mountable device, wherein said operator application comprises digitally executable instructions to render a graphical user interface (GIU) on said digital display screen and receive as input therefrom manual digital control of said dynamic visual stimulus such that a stimulus displacement on said widescreen display corresponds with a manual displacement entered via said GUI.

47.-107. (canceled)

108. The head-mountable device of claim 33, wherein said dynamic visual stimulus comprises a pattern of alternating rectangles horizontally displaced across aid widescreen display.

109. The head-mountable device of claim 33, wherein said dynamic visual stimulus comprises an alternating pattern horizontally displaced across said widescreen display.

110. The head-mountable device of claim 109, wherein said alternating pattern is horizontally displaced in performing an optokinetic nystagmus (OKN) assessment.

111. The device of claim 33, further comprising a vergence testing feature operable along an axis perpendicular to a plane of said widescreen display to present a vergence testing stimulus at a plurality of physical locations at respective relative distances to the user.

112. The device of claim 111, wherein said vergence testing feature comprises a plurality of light sources disposed along at least one of an upper or a lower viewing tunnel surface extending from above said widescreen display toward the user.

113. The device of claim 111, wherein said vergence testing feature comprises a physically displaceable stimulus.

114. The system of claim 45, wherein said viewing tunnel comprises a substantially amorphous internal surface to reduce internal reflections.

115. The system of claim 45, wherein said dynamic visual stimulus comprises an alternating pattern horizontally displaced across said widescreen display in performing an optokinetic nystagmus (OKN) assessment.
